(19) United States
(12) Patent Application Publication
(10) Pub. No.: US 2004/0023855 A1
(43) Pub. Date: Feb. 5, 2004

(54) Biologic modulations with nanoparticles

(76) Inventors: Constance M. John, San Francisco, CA (US); Gretchen Unger, Chaska, MN (US)

Correspondence Address: Kenneth I. Kohn, KOHN & ASSOCIATES, PLLC, Suite 410, 30500 Northwestern Highway, Farmington Hills, MI, 48334, US

(21) Appl. No.: 10/409,786

(22) Filed: Apr. 8, 2003

Related U.S. Application Data
(60) Provisional application No. 60/394,315, filed on Jul. 8, 2002.
(60) Provisional application No. 60/370,882, filed on Apr. 8, 2002.
(60) Provisional application No. 60/428,296, filed on Nov. 22, 2002.

(30) Foreign Application Priority Data
Jun. 10, 2002 (WO) 02/100343

(51) Int. Cl. A61K 38/17; A61K 48/00

(52) U.S. Cl. 514/8; 514/44

(57) ABSTRACT

Small particles are disclosed for use in biological systems, including the delivery of biologically active agents to cells or tissues using nanoparticles of less than about 200 nm in approximate diameter. A particle having a bioactive component, a surfactant molecule, a biocompatible polymer, and a cell recognition component, wherein the cell recognition component has a binding affinity for a cell recognition target are also disclosed. Compositions and methods of use are also set forth.

### CROSS-REFERENCE TO RELATED APPLICATIONS

This is a Continuation-In-Part application of U.S. patent application Ser. No. (not available yet) filed Feb. 28, 2003, which claims the benefit of priority of U.S. Provisional Patent Application Serial No. 60/394,315, filed Jul. 8, 2002; U.S. Ser. No. 60/370,882, filed Apr. 8, 2002; and U.S. Ser. No. 60/428,296, filed Nov. 22, 2002; as well as claiming benefit of priority of U.S. patent application Ser. No. 09/877,790 filed Jun. 8, 2001; and World Intellectual Property Organization International Publication No. WO 02/100343, filed Jun. 10, 2002, all of which are hereby incorporated herein by reference.

### BACKGROUND OF THE INVENTION

1. Field of The Invention

The field of the invention relates to the use of small particles in biological systems for the delivery of biologically active agents.

2. Description of The Prior Art

There is evidence that a class of lectins, called galectins formerly known as S-type or S-Lac lectins, play a role in inflammatory, immunomodulatory, and tumorigenic processes, in part, due to complex intercellular interactions involving carbohydrate-dependent and -independent mechanisms.

Lectins are, by definition, proteins with at least one carbohydrate-recognition domain (CRD). By immobilizing monosaccharides, oligosaccharides, or glycoproteins in affinity columns, lectins have been isolated from tissues and cells. Generally, a tissue extract in acetone or the like is prepared to isolate the protein component from the lipid component. The acetone is then evaporated, whereupon the residue is solubilized in a buffered aqueous solution. This solution is then passed through an affinity column containing the immobilized carbohydrates or glycoproteins. A number of lectins that selectively bind to galactosides have been isolated in this manner.

Galectins are a large family of carbohydrate binding proteins or lectins that are defined by a highly conserved CRD and characteristic affinity for β-galactosides (4, 15, 53, 70, 90, 111). Although most galectins bind lactose, each galectin is more specific and has higher affinity for some especially more complex saccharides. A number of reviews of the biology of galectins have been published (11, 43, 62, 68, 96, 98-101).

Galectins are unusual in that they are cytosolic proteins that lack any signal sequence for transport into the endoplasmic reticulum and are not glycosylated indicating that they do not traverse the ER-Golgi network (44). Nonethless,-galectins are localized in the extracellular matrix, and on cell surfaces, and be also be found in the nucleus of some cells. There have been 14 mammalian galectins discovered to date. These can be classified into prototype galectins (galectin-1, -2, -5, -7, -10, -11, -13, and -14) that exist as monomers or noncovalent homodimers of the carbohydrate recognition domain; chimera type galectins (galectin-3); and tandem-repeat type galectins (galectin-4, -6, -8, -9, and -12) (38). FIG. 1 illustrates this classification system for the galectin family of mammalian proteins.

Over the past several decades, active and extensive research into the use of small particles in the delivery of therapeutic macromolecules has generated a number of conventional approaches in the preparation of small particles. These approaches typically include the use of heat, high-pressure homogenization, or high intensity ultrasound sonication to prepare particles having a diameter of more than 100 nanometers, or high amounts of solvents or oils, cytotoxic chemicals, such as cross-linking agents, catalysts to prepare small particles. These approaches are challenging due to a number of variables.

For example, when organic solvents are included in the manufacturing process for small particles, the organic solvent can denature the therapeutic macromolecule that reduces most, if not all, efficacy of the therapeutic macromolecule. In fact, denaturation of the therapeutic macromolecule can even promote a toxic response upon administration of the small particle.

In addition, when an organic solvent is used to prepare small particles, the organic solvent or solvent soluble polymer can undergo degradation or other reactions that destroys the efficacy of the therapeutic macromolecule. Therefore, organic solvents can generally denature the therapeutic macromolecule during or after preparation of a small particle. As a result, organic solvents are typically removed during the manufacturing process of small particles. However, inclusion of one or more organic solvent removal techniques generally increases the costs and complexity of forming small particles. Additionally, high-pressure homogenization or high-intensity ultrasound sonication techniques often require complex and expensive equipment that generally increases costs in preparing small particles.

Therapeutic macromolecules also have limited ability to cross cell membranes. Consequently, the future success of antisense and other new molecular approaches requires innovation in drug delivery methods. Delivery of therapeutic macromolecules, particularly nucleic acids, is complicated not only by their size, but also by their sensitivity to omnipresent nuclease activity in vivo.

Therefore, there is a need for methods to prepare small particles without the use of cytotoxic chemicals or complex and expensive equipment. Additionally, a need exists to develop a small particle that can more effectively deliver antisense molecules.

### SUMMARY OF THE INVENTION

According to the prevent invention, there is provided a particle capable of delivering biologically active molecules to a cell and at least one biologically active molecule and the use of the particle for treating disease by administering the particle to a patient in need. The particles are coated with a galectin or antibody to a galectin to enhance cellular uptake particularly through caveolae, to increase the specificity of tissue or cell targeting, and to improve the process of manufacturing particles coated with cell recognition components. The biologically active molecule provided is a galectin protein, antisense to a galectin, or a nucleic acid encoding a galectin.

### BRIEF DESCRIPTION OF THE FIGURES

Other advantages of the present invention are readily appreciated as the same becomes better understood by reference to the following detailed description when considered in connection with the accompanying drawings wherein:

FIG. 1 is an illustration of the classification of the galectin family of proteins into the prototype, chimera, and tandem-repeat type groups;

FIGS. 2A and B are illustrations of one postulated mechanism of anticancer action of recombinant N-terminally truncated galectin-3 or galectin-3C; wild-type galectin-3 (FIG. 2A) cross-links cancer cells with each other and the extracellular matrix by multimerization when binding terminal β-galactosides; the N-terminally truncated protein (FIG. 2B) blocks interactions of cancer cells with β-galactosides by blocking the binding sites for wild type galectin-3;

FIG. 3 is a graph showing the in vitro cytotoxicity of nanoparticles comprising cisplatin and various cell recognition components for Alva-41, human tumor cells derived from a prostate carcinoma metastasis; and

FIGS. 4A-F are photographs showing a montage of photomicrograph showing that the treatment of Alva-41 prostate carcinoma in pig skin using nanoparticles coated with N-terminally truncated galectin-3 having casein kinase 2 alpha (CK2□) antisense sequences are able to protect the skin biopsy from overgrowth of the tumor; the nuclear stain used is bisbenzamide and the antibody to prostate specific antigen (PSA; Chemicon) was stained with biotinylated mouse IgG followed by streptavidin conjugated to Cy3.

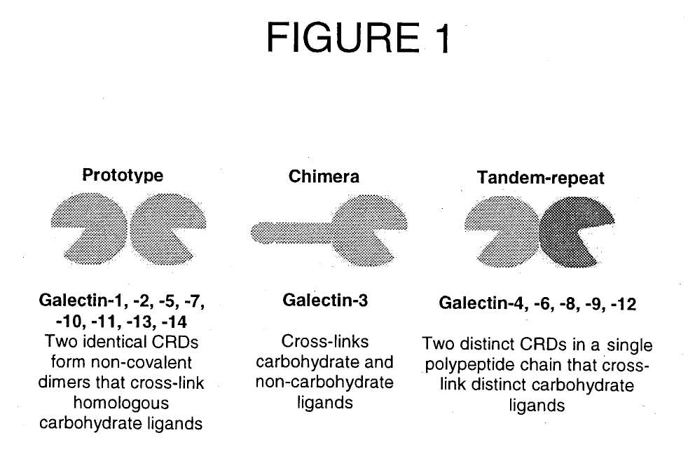

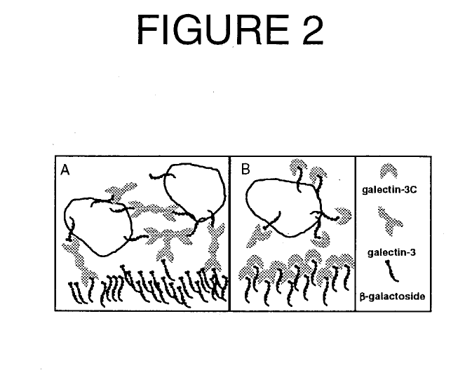

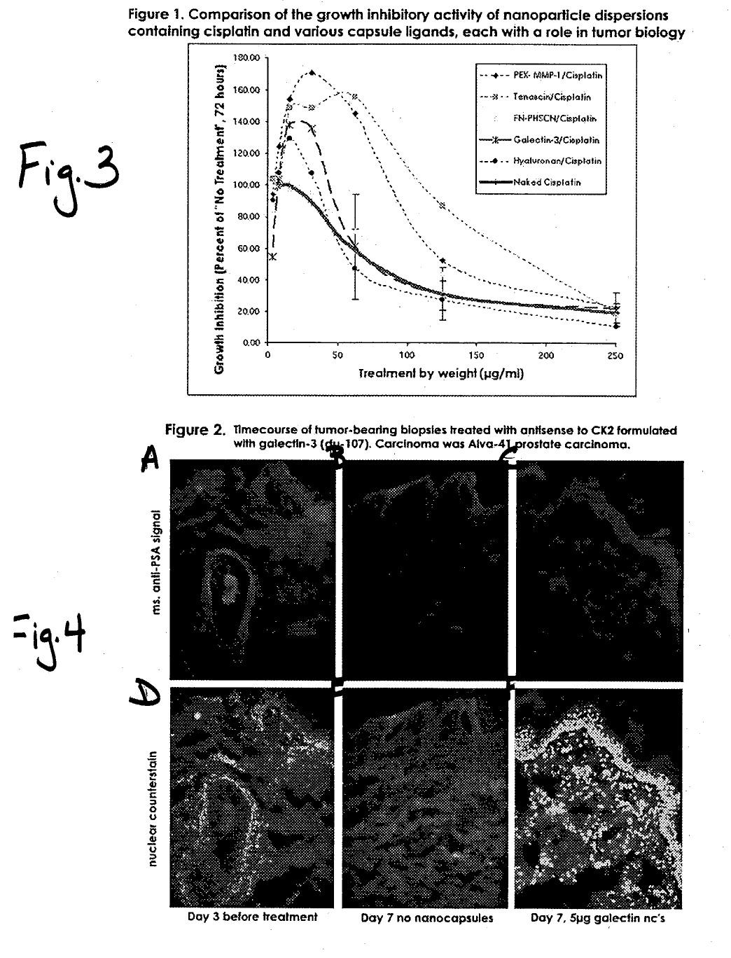

### DETAILED DESCRIPTION

Generally the present invention provides a method for delivering biologically active molecules and a nanoparticle for such delivery. The nanoparticles of the present invention can be taken up through caveolae of a cell. The caveolae are cholesterol rich vesicles that are smaller than clathrin coated pits and bypass the endosomal pathways. Entrance through caveolae is through 20-60 nanometer openings located on the surface of the target cell. Accordingly, nanoparticles are provided herein that are dimensioned to pass through caveloae, so that the nanoparticle contents are not degraded. Moreover, the nanoparticles are localized to cell nuclei after their introduction into the cell so that the nanoparticle contents are delivered in a highly effective manner that requires lower doses and concentrations than would otherwise be necessary, see copending U.S. patent application Ser. No. 09/796,575, filed Feb. 28, 2001.

More specifically, the present invention provides methods and compositions for specific delivery of macromolecules and small molecules to cell and tissue-specific targets using ligand-based nanoparticles. Embodiments include nanoparticles that can be assembled from simple mixtures of components comprising at least one ligand for a target cell surface receptor. Nanoparticles can be designed to be metastable, and/or controlled-release forms, enabling eventual release of capsule or particle contents. In one embodiment, particles are manufactured to be smaller than 50 nm enabling efficient cellular uptake by caveolar potocytosis. These particles are further distinguished by their capacity for penetration across tissue boundaries, such as the epidermis and endothelial lumen. In another embodiment, particles are manufactured to be larger than 50 nm, enabling a period of extracellular dissolution. This combined approach of using readily assembled particles with ligand-based targeting enables a method of rational design for drug delivery based on cell biology and regional administration.

Preferably, the nanoparticles are less than about 200 nm in approximately size. The particles can also include a bioactive component, a surfactant molecule, a biocompatible polymer, and a cell recognition component, wherein the cell recognition component has a binding affinity for a cell recognition target.

Examples of the bioactive component include, but are not limited to, anthracyclines, doxorubicin, vincristine, cyclophosphamide, topotecan, paclitaxel, modulators of apoptosis, growth factors, and anti-sense polynucleic acid, such as that effective to inhibit expression of CK2 polypeptides.

Examples of cell recognition targets include, but are not limited to, carbohydrates, glycoconjugate, polypeptides, and various cell adhesion molecules, members of the immunoglobulin superfamily, integrins, cadherins, selectins, growth factor receptors, collagen receptors, laminin receptors, fibronectin receptors, chondroitin sulfate receptors, dermatan sulfate receptors, heparin sulfate receptors, keratan sulfate receptors, elastin receptors, and vitronectin receptors. Alternately, the cell recognition component can be a ligand that has affinity for the cell recognition target.

Examples of cell recognition components include, but are not limited to, carbohydrates, glycoconjugates, polypeptides, and various cell adhesion molecules, members of the immunoglobulin superfamily, integrins, cadherins, selectins, growth factor receptors, collagen, laminin, fibronectin, chondroitin sulfate, dermatan sulfate, heparin sulfate, keratan sulfate, elastin, and vitronectin.

The nanoparticles can be used to deliver therapeutic compositions, including, for example, macromolecules. Without being limited to a particular theory of action, the nanoparticles are sized so as to enter through cellular caveolae and thereby overcome many of the limitations of conventional therapies. The nanoparticles enter the cell release agents that modulate cellular activity. Examples of agents are toxins, genes, and antisense DNA molecules. Other embodiments are nanoparticles that have agents for visualizing the cell, e.g., fluorescent markers or dye. Other embodiments are particles that target the exterior of a cell, or areas outside of a cell and subsequently are taken up by cells or subsequently release agents. Vontrolled release systems for controllably releasing nanoparticles for sustained delivery of the nanoparticles and agents associated with the nanoparticles are also provied. Further, methods for targeting specific cells and treating certain conditions using therapeutics delivered with nanoparticles also are provided by the present invention.

Detailed methods for making such nanoparticles are set forth in commonly owned copending U.S. patent application Ser. No. 09/796,575, filed Feb. 28, 2001. Additionally, detailed methods of making alternative forms of nanoparticles are presented herein, as well as methods of making and using the same. Certain embodiments address useful recipes for making nanoparticles, as well as therapeutic molecules for use with the same. Although the term nanoparticle is adopted herein to describe certain preferred embodiments for particles, the term includes nanoparticles and nanospheres. In general, a nanoparticle is a particle that is less than about 100 nm in average diameter, but other sizes and conformations of the nanoparticles are also contemplated.

Since nanoparticles are described herein can be capable of caveolaer cell entry, they are effective vehicles for delivering agents to cells in circumstances where conventional particles are not effective, including microparticles, liposomes, stealth liposomes, and other conventionally known particulate delivery systems, including those that have referred to as nanoparticles by others. As set forth below, nanoparticles are generally small relative to conventional particles so that delivery through the blood system and tissue is enhanced relative to conventional particle technology. The nanoparticles are generally useful for therapeutic applications, research applications, and applications in vivo, ex vivo, and in vitro.

Nanoparticles can be sized, as described herein, to enter cells via cellular caveloae, which are cholesterol-rich structures present in most cells and cell types. Entrance to these vesicles is through 20-60 nm openings. Caveolae a.k.a. plasmalemmel vesicles are small (50-80 nm), cholesterol-rich vesicles that likely derive from mobile microdomains of cholesterol in the cell membrane, a.k.a lipid rafts. These vesicles participate in a receptor-mediated uptake process known as potocytosis. Because of the lipid nature of caveolae, receptors that populate or traffic to caveolae following ligand binding typically include receptors with fatty acid tails such as GPI-linked or integrin receptors. An integral role for caveolin in mediating B-1 integrin signaling and maintenance of focal adhesions has been documented.

In contrast, the delivery of larger objects to cells is conventionally attempted using other pathways. These pathways vary in the size of molecules that they can accept. The coated pit pathway is best-known and well-studied as the pathway for receptor-mediated endocytosis. Coated pits evolve into endosomes coated with clathrin that are typically in the range of 150-200 nm. Unless a specific sorting event occurs, endosomes constitutively deliver their contents to a lysosomal vesicle for degradation.

The manufacture and process chemistry of nanoparticles is described in detail in U.S. Pat. Ser. No. 09/796,575 filed Feb. 28, 2001. In brief, a suitable method of making a nanoparticle is to form a dispersion of micelles by forming a plurality of surfactant micelles, wherein the plurality of surfactant micelles comprises a surfactant interfacing with a bioactive component, wherein the surfactant can have a hydrophile-lipophile-balance (HLB) value of less than about 6.0 units. Then the surfactant micelles are dispersed into an aqueous composition, wherein the aqueous composition comprises a hydrophilic polymer so that the hydrophilic polymer associates with the surfactant micelles to form stabilized surfactant micelles. The stabilized micelles can have an average diameter of less than about 200 or 100 or 50 nanometers. Non-ionic surfactants can alternatively be used. The stabilized surfactant micelles can be precipitated, e.g. using a cation, to form nanoparticles having an average diameter of less than about 200 or 100 or 50 nanometers, as measured by atomic force microscopy of the particles following drying of the particles. Moreover, in some embodiments, the particles can be incubated in the presence of at least one cation. Embodiments wherein nanoparticles have a diameter of less than 200 or 100 or 50 nm, including all values within the range of 5-200 nm, are contemplated. Following incubation, particles are collected by centrifugation for final processing. Particles show excellent freeze-thaw stability, stability at −4° C., mechanical stability and tolerate speed-vacuum lyophilization. Stability is measured by retention of particle size distribution and biological activity. Drug stocks of 4 mg/ml are routinely produced with 70-100% yields.

The term precipitate refers to a solidifying or a hardening of the biocompatible polymer component that surrounds the stabilized surfactant micelles. Precipitation also encompasses crystallization of the biocompatible polymer that can occur when the biocompatible polymer component is exposed to the solute. Examples of cations for precipitation include, for example, Mn2+, Mg2+, Ca2+, Al3+, Be2+, Li+, Ba2+, Gd3+.

The amount of the surfactant composition in some embodiments can range up to about 10.0 weight percent, based upon the weight of a total volume of the stabilized surfactant micelles. Typically however, the amount of the surfactant composition is less than about 0.5 weight percent, and can be present at an amount of less than about 0.05 weight percent, based upon the total weight of the total volume of the stabilized surfactant micelles. A person of ordinary skill in the art will recognize that all possible ranges within the explicit ranges are also contemplated.

A nanoparticle can be a physical structure such as a particle, nanocapsule, nanocore, or nanosphere. A nanosphere is a particle having a solid spherical-type structure with a size of less than about 1,000 nanometers. A nanocore refers to a particle having a solid core with a size of less than about 1,000 nanometers. A nanocapsule refers to a particle having a hollow core that is surrounded by a shell, such that the particle has a size of less than about 1,000 nanometers. When a nanocapsule includes a therapeutic macromolecule, the therapeutic macromolecule is located in the core that is surrounded by the shell of the nanocapsule.

Embodiments herein are described in terms of nanoparticles but are also contemplated as being performed using nanocapsules, the making and use of which are also taught in commonly assigned copending application Ser. No. 09/796,575, filed Feb. 28, 2001, which teaches methods for making particles having various sizes, including less than about 200 nm, from about 5-200 nm, and all ranges in the bounds of about 5 and about 200 nm. The same application teaches how to make s50 nanoparticles. An s50 nanoparticle is a nanoparticle that has an approximate diameter of less than about 50 nm.

The bioactive component, in some embodiments, can be partitioned from the hydrophilic polymer in the nanoparticles, and can be, for example, hydrophobic or hydrophilic. Bioactive components can include proteins, peptides, polysaccharides, and small molecules, e.g., small molecule drugs. Nucleic acids are also suitable bioactive components for use in nanoparticles, including DNA, RNA, mRNA, and including antisense RNA or DNA. When nucleic acids are the bioactive component, it is usually desirable to include a step of condensing the nucleic acids with a condensation agent prior to coating or complexing the bioactive component with the surfactant, as previously set forth in U.S. patent application Ser. No. 09/796,575, filed Feb. 28, 2001.

A wide variety of polymers can be used as the biocompatible polymer, including many biologically compatible, water-soluble and water dispersible, cationic or anionic polymers. Due to an absence of water diffusion barriers, favorable initial biodistribution and multivalent site-binding properties, hydrophilic polymer components are typically useful for enhancing nanoparticle distribution in tissues. However, it will be apparent to those skilled in the art that amphoteric and hydrophobic polymer components can also be used as needed. The biocompatible polymer component can be supplied as individual biocompatible polymers or supplied in various prepared mixtures of two or more biocompatible polymers that are subsequently combined to form the biocompatible polymer component. Though descriptions of the present invention are primarily made in terms of a hydrophilic biocompatible polymer component, it is to be understood that any other biocompatible polymer, such as hydrophobic biocompatible polymers can be substituted in place of the hydrophilic biocompatible polymer, in accordance with the present invention, while still realizing benefits of the present invention. Likewise, it is to be understood that any combination of any biocompatible polymer can be included in accordance with the present invention, while still realizing benefits of the present invention.

Antisense molecules are useful bioactive agents to deliver with nanoparticles. Nanoparticles comprising antisense molecules are typically made with a condensing agent. Some suitable nucleic acid condensing agents are poly(ethylenimine) (PEI) (at a 27,000 MW, PEI was used at about 90% charge neutralization). Polylysine (PLL) (at 7,000-150,000 molecular weight) condensing materials were conjugated with nuclear signal localization peptides, e.g., SV-40 T using carbodiimide chemistry available from Pierce Chemical (Rockford, Ill.). Preparations of nuclear matrix proteins (NMP) were collected from a rat fibroblast cell line, and a human keratinocyte cell line using a procedure described in Gerner et al. J Cell. Biochem. 71 (1998): 363-374. Protein preparations were conjugated with nuclear signal localization peptides as described.

Additional materials for use as condensation components are spermine, polyornithine, polyarginine, spermidine, VP22 protein constructs, block and graft copolymers of N-(2-hydroxypropyl)methacrylamide (HPMA) with 2-(trimethylammonio)ethyl methacrylate (TMAEM), poly[2-(dimethylamino)ethyl methacrylate], p(DMAEMA), protamine, sulfate, and peptide constructs derived from histones. Additional condensation components are known, for example as in U.S. Pat. No. 6,153,729. Antisense molecules typically require a relatively smaller condensation agent than relatively larger nucleic acid molecules. Targeting agents can also be conjugated to condensation agents, e.g., as in U.S. Pat. No. 5,922,859 and PCT Application WO/01 089579.

Nanoparticles can comprise various targeting components, e.g., ligands, to target the nanoparticle and its contents to, e.g., specific cells. The contents of the nanoparticle can be, for example, therapeutic agents that alter the activity of the cell, or a marker. The ligands can be in coatings and/or otherwise incorporated into the nanoparticles. For example, if one more than one type of cell is being cultured, a particular cell type or subset of cells can be targeted using nanoparticles having ligands that are specific to particular targets on the cells. Thus, for example, several cells in the field of view of a microscope can be observed while a subset of the cells are undergoing treatment. Thus some of the cells serve as controls for the treated cells. Or, cells can advantageously be treated while cultured with other cells, for example, some cultured stem cells are known to be advantageously grown in co-culture with other cell types. Table 1 sets forth some ligands. A ligand is a molecule that specifically binds to another molecule, which can be referred to as a target. Thus a ligand for a growth factor receptor can be, e.g., a growth factor, a fragment of a growth factor, or an antibody. Those of ordinary skill in these arts are able to distinguish specific binding from non-specific binding; for example, the identification of a ligand for a cell receptor requires distinguishing it from other molecules that nonspecifically bind the receptor.

Targeting components and/or agents delivered using nanoparticles can copolymerized, linked to, fused with, or otherwise joined or associated with other molecules, e.g., see Halin et. al, Nature Biotech. (2002) 20:264-69, “Enhancement of the antitumor activity of interleukin-12 by targeted delivery to neovasculature” for a review of fusion proteins.

Moreover, antibodies (described below) or peptides can be developed to target specific tissues. For example, a screening assay can be performed using a library and a target. Thus a library of potential ligands can be screened against targets, e.g., tumor tissue. An example of a screening method is set forth in U.S. Pat. No. 6,232,287, which describes various phage panning methods, both in vitro and in vivo. Such peptides can be incorporated into nanoparticles for targeting uses.

TABLE 1A
Targeting components for particles

| Target cell | Targeting component | Reference/Source |
|---|---|---|
| Endothelial cells | Albumin | U.S. Pat. No. 6,204,054. (for |
| | | trancytosis) |
| Keratinocytes | Laminin | Glia 8:71 |
| Tumor cells | thrombospondin (TSP) | (122) |
| | Osteopontin (OP) | (109) |
| | Thrombin-cleaved OP | |
| | Fibronectin | Unger et. al, 2001, AAPS Pharmsci 3(3) |
| | | Supplement: 3731 |
| Myocytes | Fibronectin, Laminin | (39) |
| | β1d integrin ligands | Am. J. Phys. 279(6): H2916-26 |
| | PVP 10,000 MW | |
| hepatocytes/liver | DGEA peptide | (112) |
| cells | | |
| hepatic stellate | Collagen, laminin | Gastroent 110: 1127-1136 |
| chondrocytes/bone | Osteopontin | Cell Ad Commun 3:367-374, US |
| cells | | 6074609, US 5770565, PCT W0 |
| | | 0980837A1, PCT W0 0209735A2 |
| | BMP | U.S. Pat. No. 6352972 |
| | SPARC/osteonectin | PCT W0 072679a1 |
| | collagen2 | PCT W0 145764a1 |
| | HA | U.S. Pat. Nos. 51,283,26 & |
| | | 5,866,165 |
| | Osteocalcin | U.S. Pat. No. 6,159,467 |
| Smooth muscle | Osteopontin | U.S. Pat. No. 5849865 |
| cells | | |
| Stem cells | FN, rE-selectin, HA | (55) |
| Neurons | Nerve Growth Factor, | Development 124 (19): 3909-3917 |
| | Agrin | |
| | contactin ligand | U.S. Pat. No. 5766922 |
| | NCAM, L1 | U.S. Pat. No. 5792743 |
| | KAL | U.S. Pat. No. 6121231 |
| | Phosphacan | U.S. Pat. No. 5625040 |
| | Neurocan | U.S. Pat. No. 5648465 |
| | Cytotactin | U.S. Pat. No. S 6482410 |
| | Laminin, KS- and β1k | U.S. Pat. No. 5,610,031 |
| | chain | U.S. Pat. No. 5,580,960 |
| | Merosin | U.S. Pat. No. 5,872,231 |
| Schwann | Ninjurin | U.S. Pat. No. 6,140,117 |
| cells/neuron | | |
| Retinal ganglion | Osteonectin | J. Histochem Chem 46(1):3-10 |
| | Laminin | Dev. Biol. 138:82-93 |
| Muller cells | rNcam,r L-1 rN- | Dev. Biol. 138(1):82-93 |
| | cadherin | |
| Blood-Brain barrier | Peptide vectors e.g. d- | Rouselle et. al, Molecular |
| | penetratin, pegelin, | Pharmacology, (2000) 57:679-686 |
| | protegrins and related | |

TABLE 1B
Additional Candidate Excipients for angiogenic and anti-tumor
particle targeting agents

| Candidate Particle | Potential Role in Tumor | |
|---|---|---|
| Material | Biology | Reference |
| Recombinant Pex binding | Extravasation of tumor | (6) |
| domain of membrane- | cells from bloodstream into | |
| associated Matrix | distant site from primary | |
| Metalloproteinase -1 | tumor | |
| Bovine bone-derived | Chemokine attracting | (52) |
| Osteonectin | metastatic tumor cells to | |
| | bone | |
| Fibronectin inhibitory | Blocks □5□1 integrin | (71) |
| peptide, PHSCN | binding site on migrating | |
| | tumor cells, preventing | |
| | tissue extravasation | |
| Recombinant truncated | Modified ligand for CEA | PCT WO 02100343A2 |
| Galectin-3 | antigen, plays role in | (30) |
| | tumor cell extravasation | |
| Hyaluronan | Feature of tumor stroma, | (110) |
| | plays role in tumor | |
| | extravasation | |
| Tenascin | Feature of tumor stroma | (117) |

The nanoparticles and particles can include ligands that bind to cellular adhesion molecules and thereby target the nanoparticle and its contents to specific cells. Various cell surface adhesion molecules are active in numerous cellular processes that include cell growth, differentiation, development, cell movement, cell adhesion, and cancer metastasis. There are at least four major families of cell adhesion molecules: the immunoglobulin (Ig) superfamily, integrins, cadherins, and selectins. Cell adhesion molecules are critical to numerous cellular processes and responses. Additionally, they also play a role in various disease states. For example, tumorigenesis is a process that involves cell adhesion molecules. For successful tumorigenesis, there must be changes in cellular adhesivity that facilitate the disruption of normal tissue structures. Cell adhesion molecules are objects of intense study and improved tools for use with these molecules are required for in vitro and in vivo applications.

Members of the Ig superfamily include the intercellular adhesion molecules (ICAMs), vascular-cell adhesion molecule (VCAM-1), platelet-endothelial-cell adhesion molecule (PECAM-1), and neural-cell adhesion molecule (NCAM). Each Ig superfamily cell adhesion molecule has an extracellular domain, which has several Ig-like intrachain disulfide-bonded loops with conserved cysteine residues, a transmembrane domain, and an intracellular domain that interacts with the cytoskeleton. The Ig superfamily cell adhesion molecules are calcium-independent transmembrane glycoproteins.

Integrins are transmembrane proteins that are constitutively expressed but require activation in order to bind their ligand. Many protein and oligopeptide ligands for integrins are known. Integrins are non-covalently linked heterodimers having alpha (□ and beta (□) subunits. About 15 □ subunits and 8 □subunits have been identified: These combine promiscuously to form various types of integrin receptors but some combinations are not available, so that there are subfamilies of integrins that are made of various □ and □combinations. Integrins appear to have three activation states: basal avidity, low avidity, and high avidity. Additionally, cells will alter integrin receptor expression depending on activation state, maturity, or lineage.

The cadherins are calcium-dependent adhesion molecules and include neural (N)-cadherin, placental (P)-cadherin, and epithelial (E)-cadherin. All three belong to the classical cadherin subfamily. There are also desmosomal cadherins and proto-cadherins. Cadherins are intimately involved in embryonic development and tissue organization. They exhibit predominantly homophilic adhesion, and the key peptidic motifs for binding have been identified for most cadherins. The extracellular domain consists of several cadherin repeats, each is capable of binding a calcium ion. Following the transmembrane domain, the intracellular domain is highly conserved. When calcium is bound, the extracellular domain has a rigid, rod-like structure. The intracellular domain is capable of binding the a, b, and g catenins. The adhesive properties of the cadherins have been shown to be dependent upon the ability of the intracellular domain to interact with cytoplasmic proteins such as the catenins.

The selectins are a family of divalent cation dependent glycoproteins that bind carbohydrates, binding fucosylated carbohydrates, especially, sialylated Lewisx, and mucins. The three family members include: Endothelial (E)-selectin, leukocyte (L)-selectin, and platelet (P)-selectin. The extracellular domain of each has a carbohydrate recognition motif, an epidermal growth factor (EGF)-like motif, and varying numbers of a short repeated domain related to complement-regulatory proteins (CRP). Each has a short cytoplasmic domain. The selectins play an important role in aspects of cell adhesion, movement, and migration.

TABLE 2
Examples of Cell Recognition Components Specific for Cell
Recognition Targets

| | Alternative | | |
|---|---|---|---|
| Targeting | Names | | Example of Tumor |
| Ligands | (trade name) | Target | Target |
| RGD peptide | | Cellular adhesion | Vasculature |
| | | molecules, such as | endothelial cells in |
| | | integrins | solid tumors |
| NGR | | Aminopeptidase N | Vasculature |
| | | (CD13) | endothelial cells in |
| | | | solid tumors |
| Folate | | Folate receptor | Cancer cells that |
| | | | overexpress the folate |
| | | | receptor |
| Transferrin | | Transferrin receptor | Cancer cells that |
| | | | overexpress the |
| | | | transferrin receptor |
| GM-CSF | | GM-CSF receptor | Leukaemic blasts |
| Galactosamine | | Galactosamine | Hepatoma |
| | | receptors on | |
| | | hepatocytes | |
| Anti-VEGFR | 2C3 | Vasculature endothelial | Vasculature |
| antibody | | growth-factor receptor | endothelial cells in |
| | | (FLK1) | solid tumors |
| Anti-ERBB2 | Trastuzumab | ERBB2 receptor | Cells that overexpress |
| antibody | (Herceptin) | | the ERBB2 receptor, |
| | | | such as in breast and |
| | | | ovarian cancers. |
| Anti-CD20 | Rituximab | CD20, a B-cell surface | Non-Hodgkin's |
| antibody | (Rituxan), | antigen | lymphoma and other |
| | ibritumomab | | B-cell |
| | tiuxetan | | lymphoproliferative |
| | (Zevalin) | | diseases |
| Anti-CD22 | Epratuzumab, | CD22, a B-cell surface | Non-Hodgkin's |
| antibody | LL2, RFB4 | antigen | lymphoma and other |
| | | | B-cell |
| | | | lymphoproliferative |
| | | | diseases |
| Anti-CD19 | B4, HD37 | CD19, a pan-B-cell | Non-Hodgkin's |
| antibody | | surface epitope | lymphoma and other |
| | | | B-cell |
| | | | lymphoproliferative |
| | | | diseases |
| Anti-CD33 | Gemtuzumab, | CD33, a sialo-adhesion | Acute myeloid |
| antibody | ozogamicin | molecule, leukocyte | leukemia |
| | (Mylotarg) | differentiation antigen | |
| Anti-CD33 | M195 | CD33, a T-cell epitope | Acute myeloid |
| | | | leukemia |
| Anti-CD25 | Anti-Tac, LMB2 | CD25, subunit of the | Hairy-cell leukaemia, |
| | | interleukin-2 receptor | Hodgkin's and other |
| | | on activated T cells | CD25⁺ lymphoma |
| | | | haematological |
| | | | malignancies |
| Anti-CD25 | Denileukin | Interleukin-2 receptor | Cutaneous T-cell |
| | diftitox (Ontak) | | lymphoma |
| Anti-HLA-DR10 | Lym1 | HLA-DR10 subunit | Non-Hodgkin's |
| | | | lymphoma and other |
| | | | B-cell |
| | | | lymphoproliferative |
| | | | diseases |
| Anti-tenascin | 81C6 | Extracellular-matrix | Glial tumors, breast |
| | | protein overexpressed | cancer |
| | | in many tumors | |
| Anti-CEA | MN-14, F6, | CEA | Colorectal, small-cell |
| | A5B7 | | lung and ovarian |
| | | | cancers |
| Anti-MUC1 | HMFG1, BrE3 | MUC1, an aberrantly | Breast and bladder |
| | | glycosylated epithelial | cancer |
| | | mucin | |
| Anti-TAG72 | CC49, B72.3 | TAG72, oncofetal | Colorectal, ovarian |
| | | antigen tumor- | and breast cancer |
| | | associated | |
| | | glycoprotein-72 | |

The nanoparticles also can include growth factors so that the nanoparticles are specifically targeted to cells expressing the growth factor receptors. Other embodiments include nanoparticles having growth factors that are delivered to the cell to modulate the activity of the cell. Other embodiments include ligands that specifically bind to growth factor receptors so as to specifically target the nanoparticle to cells having the growth factor receptor.

Growth factors are active in many aspects of cellular and tissue regulation including proliferation, hyperproliferation, differentiation, trophism, scarring, and healing, as shown in, for example, Table 3. Growth factors specifically bind to cell surface receptors. Many growth factors are quite versatile, stimulating cellular activities in numerous different cell types; while others are specific to a particular cell-type. Targeting nanoparticles to a growth factor receptor enables the activity of the cell to be controlled. Thus many aspects of physiological activity can be controlled or studied, including proliferation, hyperproliferation, and healing. A growth factor refers to a growth factor or molecules comprising an active fragment thereof, and includes purified native polypeptides and recombinant polypeptides.

Nanoparticles can be targeted to growth factor receptors by a variety of means. For example, antibodies against the receptor can be created and used on the nanoparticles for direction specifically to the receptor. Or, the growth factor, or a fragment thereof, can be used on the nanoparticles to direct specifically to the receptor. The blinding of growth factors to growth factor receptors has, in general, been extensively studied, and short polypeptide sequences that are a fragment of the growth factors, and bind to the receptors, are known.

For example, if it is desirable to limit the proliferation of glial or smooth muscle cells, a particle associated with a cell behavior modulating agent, e.g., a toxin or antiproliferative agent, can be decorated with a ligand that specifically binds PDGF-R (Table 3). Since PDGF-R is preferentially expressed by glial or smooth muscle cells, the particles will preferentially be taken up by glial or smooth muscle cells. The toxin would kill the cells or the antiproliferative agent would reduce proliferation. Similarly, specifically specifically targeting nanoparticles having modulating agents can control other cellular activities, such as those set forth in Table 3.

TABLE 3
Growth Factors and Growth Factor Receptors
for Cell and Tissue Targeting

| Factor | Receptor | Source | Activity | Comments |
|---|---|---|---|---|
| PDGF | PDGF-R | platelets, | proliferation of | two different |
| | | endothelial | connective | protein chains |
| | | cells, placenta | tissue, glial and | form 3 distinct |
| | | | smooth muscle | dimer forms; |
| | | | cells | AA, AB and BB |
| EGF | EGF-R | submaxillary | proliferation of | |
| | | gland, | mesenchymal, | |
| | | Brunners | glial and | |
| | | gland | epithelial cells | |
| TGF-a | TGF-a-R | common in | active for | related to EGF |
| | | transformed | normal wound | |
| | | cells | healing | |
| FGF | FGF-R | wide range of | promotes | at least 19 |
| | | cells; protein is | proliferation of | family |
| | | associated | many cells; | members, 4 |
| | | with the ECM | inhibits some | distinct |
| | | | stem cells | receptors |
| NGF | NGF-R | | promotes | related proteins |
| | | | neurite | identified as |
| | | | outgrowth and | proto- |
| | | | neural cell | oncogenes; |
| | | | survival | trkA, trkB, trkC |
| Erythropoietin | Erythropoietin- | kidney | promotes | |
| | R | | proliferation | |
| | | | and | |
| | | | differentiation | |
| | | | of erythrocytes | |
| TGF-b | TGF-b-R | activated TH₁ | anti- | at least 100 |
| | | cells (T-helper) | inflammatory, | different family |
| | | and natural | promotes | members |
| | | killer (NK) cells | wound healing, | |
| | | | inhibits | |
| | | | macrophage | |
| | | | and lymphocyte | |
| | | | proliferation | |
| IGF-I | IGF-I-R | primarily liver | promotes | related to IGF-II |
| | | | proliferation of | and proinsulin, |
| | | | many cell types | also called |
| | | | | Somatomedin C |
| IGF-II | IGF-II-R | variety of cells | promotes | related IGF-I |
| | | | proliferation of | and proinsulin |
| | | | many cell types | |
| | | | primarily of | |
| | | | fetal origin | |

Epidermal growth factor (EGF), like all growth factors, binds to specific high-affinity, low-capacity cell surface receptors. Intrinsic to the EGF receptor is tyrosine kinase activity, which is activated in response to EGF binding. EGF has a tyrosine kinase domain that phosphorylates the EGF receptor itself (autophosphorylation) as well as other proteins, in signal transduction cascades. Experimental evidence has shown that the Neu proto-oncogene is a homologue of the EGF receptor, indicating that EGF is active in cellular hyperproliferation. EGF has proliferative effects on cells of both mesodermal and ectodermal origin, particularly keratinocytes and fibroblasts. EGF exhibits negative growth effects on certain carcinomas as well as hair follicle cells. Growth-related responses to EGF include the induction of nuclear proto-oncogene expression, such as Fos, Jun and Myc.

Fibroblast Growth Factors (FGFs) are a family of at least 19 distinct members. Kaposi's sarcoma cells (prevalent in patients with AIDS) secrete a homologue of FGF called the K-FGF proto-oncogene. In mice the mammary tumor virus integrates at two predominant sites in the mouse genome identified as Int-1 and Int-2. The protein encoded by the Int-2 locus is a homologue of the FGF family of growth factors. A prominent role for FGFs is in the development of the skeletal system and nervous system in mammals. FGFs also are neurotrophic for cells of both the peripheral and central nervous system. Additionally, several members of the FGF family are potent inducers of mesodermal differentiation in early embryos. The FGFs interact with specific cell-surface receptors that have been identified as having intrinsic tyrosine kinase activity. The FIg proto-oncogene is a homologue of the FGF receptor family. FGFR3 is predominantly expressed in quiescent chondrocytes where it is responsible for restricting chondrocyte proliferation and differentiation. In mice with inactivating mutations in FGFR3 there is an expansion of long bone growth and zones of proliferating cartilage further demonstrating that FGFR3 is necessary to control the rate and amount of chondrocyte growth.

Platelet-Derived Growth Factor (PDGF) has two distinct polypeptide chains, A and B. The c-Sis proto-oncogene has been shown to be homologous to the PDGF A chain. Like the EGF receptor, the PDGF receptors have autophosphorylating tyrosine kinase activity. Proliferative responses to PDGF action are exerted on many mesenchymal cell types. Other growth-related responses to PDGF include cytoskeletal rearrangement and increased polyphosphoinositol turnover. PDGF induces the expression of a number of nuclear localized proto-oncogenes, such as Fos, Myc and Jun.

Transforming Growth Factors-beta (TGFs-beta) was originally characterized as a protein (secreted from a tumor cell line) that was capable of inducing a transformed phenotype in non-neoplastic cells in culture, and thus is implicated in numerous hyperproliferation disorders. The TGF-beta-related family of proteins includes the activin and inhibin proteins. The Mullerian inhibiting substance (MIS) is also a TGF-beta-related protein, as are members of the bone morphogenetic protein (BMP) family of bone growth-regulatory factors. Indeed, the TGF-beta family can comprise as many as 100 distinct proteins, all with at least one region of amino-acid sequence homology. There are several classes of cell-surface receptors that bind different TGFs-beta with differing affinities. The TGF-beta family of receptors has intrinsic serine/threonine kinase activity and, therefore, induces distinct cascades of signal transduction. TGF-betas have proliferative effects on many mesenchymal and epithelial cell types and sometimes demonstrate anti-proliferative effects on endothelial cells.

Transforming Growth Factor-a (TGF-alpha) was first identified as a substance secreted from certain tumor cells that, in conjunction with TGF-alpha-1, could reversibly transform certain types of normal cells in culture, and thus is implicated in numerous hyperproliferative disorders. TGF-alpha binds to the EGF receptor, as well as its own distinct receptor, and it is this interaction that is thought to be responsible for the growth factor's effect. The predominant sources of TGF-alpha are carcinomas, but activated macrophages and keratinocytes (and possibly other epithelial cells) also secrete TGF-alpha. In normal cell populations, TGF-alpha is a potent keratinocyte growth factor.

Tumor Necrosis Factor-alpha (TNF-alpha) TNF-alpha (also called lymphotoxin) is characterized by its ability to kill a number of different cell types, as well as the ability to induce terminal differentiation in others. One significant non-proliferative response to TNF-alpha is an inhibition of lipoprotein lipase present on the surface of vascular endothelial cells. The predominant site of TNF-alpha synthesis is T-lymphocytes, in particular the special class of T-cells called cytotoxic T-lymphocytes (CTL cells). The induction of TNF-alpha expression results from elevations in IL-2 as well as the interaction of antigen with T-cell receptors.

The nanoparticles can also be associated with extracellular matrix molecules so that the particles are specifically targeted to cells expressing receptors for the extracellular matrix molecules. Alternatively, particles can comprise ligands for the extracellular matrix molecules so that the particles become associated with the extracellular matrix molecules on tissues or cells.

The extracellular matrix comprises a variety of proteins, glycoconjugates, glycoproteins, and polysaccharides that are assembled into organized matrices that form the scaffold of tissues. The common components of the extracellular matrix can be referred to as extracellular matrix molecules. Examples of extracellular matrix molecules are tenacin, collagen, laminin, fibronectin, hyaluronic acid, chondroitin sulfate, dermatan sulfate, heparin sulfate, heparin, keratan sulfate, elastin, vitronectin, and subtypes thereof. Cells typically secrete extracellular matrix molecules in response to their environments, so that the patterns of extracellular matrix molecule expression can be indicative of certain conditions. For example, EDA, a domain of fibronectin can be targeted for cancer.

Nanoparticles targeted to the extracellular matrix are useful for variety of therapeutic, scientific, and research applications. For example, extracellular matrix molecules specifically bind to receptors on cells, so that nanoparticles comprising extracellular matrix molecules are thereby targeted to extracellular matrix molecule receptors. Further, drugs can be targeted to the extracellular matrix by making nanoparticles having ligands and/or coatings that bind extracellular matrix molecules. Moreover, particles having visualization agents directed to extracellular matrix molecules can be used for microscopy, e.g. fluorescence or histochemistry.

Aberration in the patterns of expression of extracellular matrix molecules can indicate pathological conditions. For example, human tenascin is an extracellular matrix molecule, a 240.7 kDa glycoprotein. Tenascin is found in abundance in embryonic tissue, whereas the expression in normal adult tissue is limited. Tenascin has been reported to be expressed in the stroma of many tumors, including gliomas, breast, squamous cell and lung carcinomas. Thus it is possible to control hyperproliferative conditions, including many tumors, by specifically directing therapeutic agents to tenacin.

Tenascin is an extracellular matrix molecule that is useful for nanoparticles. Tenascin is a branched, 225 KD fibronectin-like (FN) extracellular protein prominent in specialized embryonic tissues, wound healing and tumors. The appearance of tenascin-C surrounding oral squamous cell carcinomas appears to be a universal feature of these tumors, while tenascin-rich stroma has been consistently observed adjacent to basal cell, esophageal, gastric, hepatic, colonic, glial and pancreatic tumor nests. Production of TN by breast carcinoma cells and stromal fibroblasts correlates with increased invasiveness. In the adult, normal cells aside from wound-activated keratinocytes, do not migrate on tenascin. However, integrin receptors capable of mediating migration on TN by carcinoma cells include αᵥβ₁, αᵥβ₃, and αᵥβ₆. Based on this information, it was hypothesized that TN nanoparticles could deliver nucleic acids specifically via receptor-mediated caveolar endocytosis.

Tenascin has been implicated in cancer activities and also as being specific for smooth muscle cells; furthermore, peptidic domains of tenascin have been identified e.g., as in U.S. Pat. No. 6,124,260. Moreover, tenascin peptides and domains have adhesion with particular cell types and the interaction between smooth muscle cells and tenascin-C has been elucidated. It is believed that the interaction between smooth muscle cells and the Fbg-L domain of tenascin-C is involved in cell adhesion and migration, and blocking this interaction would blunt SMC migration from media into the neointima and thereby affect neointimal formation.

Hyaluronan is also an extracellular matrix molecule that is useful for nanoparticles. Hyaluronan is preferentially expressed by hepatocytes and has been implicated angiogenesis. It is available in a variety of forms and has many known uses, e.g., as in U.S. Pat. No. 5,902,795.

The nanoparticles also can include galectins as the cell recognition component that target the nanoparticles to cells expressing the carbohydrate ligands specific for the galectin, and as the bioactive molecule. By including an antibody to a galectin as the cell recognition component the nanoparticle can be targeted to specific tissues and cells that express the galectin. The bioactive galectin component can be a galectin polypeptide, the nucleic acid encoding for a galectin, or an anti-sense DNA compound that inhibits expression of a galectin. To date, 14 members of the galectin family of mammalian proteins have been identified (16, 102) but it is expected that as many as 6 or so more can be isolated in man (62).

The galectins possess a number of characteristics that make them uniquely valuable as ligand coatings for nanocapules. These include the following: (a) enhanced cellular uptake via the inherent property of the galectins for stimulation of their own endocytosis into the cytoplasm or into the cytoplasm and nucleus of some cells, and trancystosis and shuttling from one cell into another (18, 23, 44, 66, 67); (b) the inherent bioactivity of specific galectins that would be beneficial in cancer, inflammatory states, and other disease; (c) the ability to target galectin-coated nanocapsules to specific tissues or cells based on varying affinity for specific glycoconjugates;. (d) and comparative ease of production of galectins in bacterial cell culture due to their relatively small size and lack of glycosylation (44). Since the expression of particular galectins is limited to certain cells, the variable distribution of a specific galectin can be targeted with an antibody or an antibody fragment to achieve targeting of a nanoparticle to a specific type of cell or organ.

There have been several studies exploring the mechanism of the non-classical secretion of galectins. Recently, galectin-3 has been shown to be able to stimulate its own endocytosis by caveolae in a concentration and lactose dependent manner (23). Galectin-3 is among the best studied of the galectins, and it is likely that the mechanism for secretion and endocytosis of galectin-3 is common to that of the entire family. Thus, a fourth advantage of using galectins to coat nanoparticles is that the cellular uptake of nanoparticules through caveolae should be enhanced due to the affinity of galectins for caveolae.

A number of reviews of the biology of galectins have been published (11, 43, 62, 68, 96, 98-101). Galectins in general possess affinity for galactose, including lactose and N-aceyllactosamine, but greater affinity for certain oligosaccharides. A detailed analysis of sugar binding specificity of a number of galectins and galectin CRDs was performed with frontal affinity chromatography (38) and revealed significant differences.

TABLE 4
Galectins for Cell and Tissue Targeting

| | | | Classification & |
|---|---|---|---|
| Factor | Distribution | Activity | References |
| Galectin-1 | Placenta, ovary, testis, | Induces apoptosis in immature | Prototype (5, |
| | liver, lymph node, thymus, | thymocytes, activated | 10, 95) |
| | spleen, macrophages, T | periperhal T cells, infected | |
| | cells, B cells | macrophages; decreases | |
| | | production of TNF-□, II-2, IFN- | |
| | | □ in activated T cells, and II-12 | |
| | | from infected macrophages, | |
| | | increases release of IL-5 | |
| Galectin-2 | Stomach epithelial cells | | Prototype |
| Galectin-3 | Macrophages, epithelial | Reduces apoptosis in activated | Chimera type |
| | cells, fibroblast, activated | T cells, macrophages, and | (3, 48, 76, 82) |
| | T cells, tumors | tumor cells, increases mast cell | |
| | | activation and superoxide | |
| | | production by neutrophils, | |
| | | decreases release of IL-5 by | |
| | | eosinophils | |
| Galectin-4 | Gastrointestinal tract | Expressed at sites of tumor cell | Tandem-repeat |
| | | adhesion | type |
| Galectin-5 | Erythrocytes | | Prototype (27) |
| Galectin-6 | Gastrointestinal tract | | Tandem-repeat |
| | | | type (28) |
| Galectin-7 | Keratinocytes, carcinoma | Increases apoptosis of | Prototype (56, |
| | | keratinocytes | 64, 73, 74) |
| Galectin-8 | Liver, kidney, cardiac | Increases apoptosis in lung | Tandem-repeat |
| | muscle, lung, prostate, | carcinoma, modulates integrin | type(8, 65) |
| | brain | interactions with the | |
| | | extracellular matrix | |
| Galectin-9 | Thymus, kidney, | Increases apoptosis in | Tandem-repeat |
| | Hodgkin's lymphoma | immature thymocytes | type |
| Galectin- | Eosinophils and basophils | Lyophospholipase activity | Prototype, |
| 10 | | | binds mannose |
| | | | not β- |
| | | | galactosides |
| | | | (2, 104, 113) |
| Galectin- | Lens | | Prototype, like |
| 11 | | | galectin-10 |
| | | | lacks affinity for |
| | | | β-galactosides |
| | | | (91) |
| Galectin- | Adipocytes, carcinoma | Increases apoptosis of | Tandem-repeat |
| 12 | | adipocytes | type (40) |
| Galectin- | Placenta | | Prototype (125) |
| 13 | | | |
| Galectin- | Eosinophils | | Prototype (21) |
| 14 | | | |

Other targeting coatings useful for nanoparticles besides galectins include hyaluronan, osteonectin, and tenasin. However, hyaluronan is a large glycosaminoglycan. The mass of the recombinant hyaluronan used in this study was 1 million kilo Dalton (kD) (114). Other coatings employed included tenascin that is large recombinant glycoprotein of 220 kD (50, 84), and osteonectin, a 31 kD glycoprotein (124). Bacteria do not glycosylate proteins, thus, mammalian cell culture would have to be employed to produce these glycoproteins. Galectins are not glycosylated, range in size from about 15 to 35 kD, and most galectins can be easily produced as recombinant proteins from cultures of Escherichia coli bacteria. In general, it is less expensive to produce recombinant proteins in bacterial culture compared to mammalian cell culture. Affinity chromatography using media such as lactosyl-Sepharose can be used to purify the proteins from the bacteria.

There is close homology between the galectin-3 proteins of different species, but the number of N-terminal tandem repeats and, hence, the sizes of the proteins vary. Galectin-3 is unique compared to other members of the galectin family in that it contains in addition to the CRD on the C-terminus an unrelated non-carbohydrate-binding N-terminal domain of 120 amino acids (human protein). Galectin-3 is isolated as a monomer but undergoes multimerization on binding to surfaces that contain glycoconjugate ligands and cross linking them. The N-terminal half of the protein primarily produces the homophilic interactions involved in cross-linking (69, 75). The carbohydrate recognition domain can form dimers but these occur in competition with carbohydrate binding (127).

Galectin-3 in adults is expressed in activated macrophages, basophils, mast cells, polarized epithelial cells, and sensory neurons, and in other cells during embryogenesis. Galectin-3 can be found on the plasma membrane, and depending on the cell type is limited to the cytoplasm or found in both the cytoplasm and nucleus (26, 32, 121). Loss of nuclear localization of galectin-3 was observed in senescent human fibroblasts (93). Binding to synexin was found to be associated with nuclear transport of galectin-3 (129). In galectin-3 deficient mice dramatically different cell morphology was observed in macrophages that were more prone to undergo apoptosis (42).

Galectin-3 can be secreted by a nonclassical mechanism (67, 78) and internalized by cells (67). Galectin-3 shares the ability to be secreted despite the absence of a signal peptide with a number of other proteins that have unconventional intercellular transfer. These proteins are internalized by cells and are able to directly access the cytoplasm and the nucleus by a process that does not involve classical endocytosis (97). This is in contrast with the modulation of intercellular events by second messengers that bind to extracellular receptors and initiate a cascade of intracellular events that often involve transciptional regulation. Although the mechanisms for the ability of some proteins to cross biological membranes in the absence of a signal sequence are poorly understood, a number of common features have been identified. Many of the proteins can directly access the nucleus, their mechanisms for secretion. In Transwell culture chambers more than 90% of apically bound radiolabeled exogenous galectin-3 was endocytosed by dog-kidney epithelial cell line MDCK II (Madin-Darby Canine Kidney) and 20% of galectin-3 was transcytosed after 120 min. The endocytosis and transcytosis were lactose-inhibitable (66). Recent work indicates that galectin-3 stimulates its own endocysis along with □1 integrins to which it binds via caveolae in a concentration dependent process (23).

Galectin-3 has been shown to bind to a number of glycosylated substrates that are implicated in tumorigenicity and metastasis (see Table 5). The affinity of galectin-3 and the N-terminally truncated galectin-3 formed by digestion with Clostridium histiolyticum collagenase type VlI (galectin-3C) was higher for a panel of oligosaccharides than other members of the galectin family (38). Laminin is the major non-collagenous polypeptide of basement membranes, and galectin-3 binds preferentially to mouse tumor laminin compared to human placental laminin (87). Other reported ligands of galectin-3 include polylactosaminoglycan (111), Mac-2 binding protein (47), IgE (115), appropriately glycosylated forms of fibronectin(107), lysosomal-membrane associated glycoproteins (Lamps) 1 and 2 (106), alpha 1 beta 1 integrin, the tumor-associated carbohydrate Thomsen-Friedenreich antigen (T antigen) (31), hensin, a protein that is involved in terminal differentiation of epithelial cells (37), advanced glycation end (AGE) products (130), carcinoembryonic antigen (34, 92) and N-acetyllactosaminylated Mgat5 glycans (19, 20). Galectin-3 has been found to bind to both single-stranded DNA and RNA in the nucleus and this binding was not inhibitable by lactose (121).

Galectin-3 binds to three glycoproteins that are implicated in tumorigenicity and metastasis, carcinoembryonic antigen (CEA), lysosome-associated membrane glycoproteins (60), and laminin. Galectin-3 previously had been reported to bind to laminin expressed by human colon cancer cells (60). In the study by Ohannesian et aL coimmunoprecipitation and binding to immobilized galectin-3 were used to isolate its ligands and the identities of the affinity-purified material were confirmed by Western blots and immunoprecipitation with specific antibodies. In addition, galectin-3 was colocalized with CEA on the surface of KM12 colon cancer cells (92).

CEA is a tumor marker that is upregulated in many kinds of cancer including colon cancer (94). In colorectal cancer preoperative serum CEA levels have been found to be a prognostic factor in node-negative colon cancer patients who undergo curative resection (34). CEA is thought to be involved in the metastatic process by its homo-and heterophilic binding properties (123), and is able to inhibit apoptosis induced by the loss of anchorage of colon cancer cells to the extracellular matrix.

Lysosome-associated membrane glycoproteins (LAMPs) have been found to be expressed more intensely in the epithelium of colorectal cancers than in normal mucosa (P<0.05) (24). Similarly, highly metastatic cells in sublines of a human colon cancer were found to express more lysosome-associated membrane glycoproteins-1 and -2 on their surface (105). LAMP-1 is a major component of lysosomal membranes that could be involved in the process of programmed cell death as it is upregulated in glioblastoma cells that have been induced to undergo apoptosis (14).

Laminins are the major non-collagenous polypeptide of basement membranes and have been implicated in the progression of carcinomas (72). Expression of laminin-5 gamma 2 chain has been correlated with unfavorable prognosis in colon cancer (63). Galectin-3 has been found to bind to laminin in several different cell and tumor types (13, 57, 60, 88).

Galectin-3 has been shown to increase the binding of cancer cells to extracellular matrix proteins (87, 89). In addition to increasing the binding of tumor cells to basement membranes, the interaction of cell surface galectin-3 with complementary serum glycoproteins appears to promote aggregation of tumor cells in circulation and thereby play a role in the pathogenesis of metastasis.

Studies of the expression of galectin-3 have found a positive correlation with some types of cancer and tumorigenicity and metastasis such as in colon (81, 108) and thyroid cancer (49); studies of other cancers have indicated there is a negative correlation (12, 46). In prostate cancer loss of the nuclear expression of galectin-3 was associated with malignancy (119). Correlation of galectin-3 expression and tumorigenicity has been complicated by its expression in normal epithelial cells that in give rise to some cancers, and the lack of knowledge of the significance of the variable localization of galectin-3. A study that focused on analysis of the extracellular expression of galectin-3 found that the concentrations in the sera of 99 patients with breast, gastrointestinal, lung, ovarian cancer, melanoma, or non-Hodgkin's lymphoma were elevated compared to the concentrations in sera of normal patients. In addition, patients with metastatic disease had higher levels than individuals that had localized tumors (51).

Expression of recombinant galectin-3 in weakly metastatic fibrosarcoma cells resulted in an increased incidence of experimental lung metastases in syngeneic and nude mice (103). In human umbilical vein endothelial cells (HUVEC) galectin-3 induces angiogenesis (82). Exogenous galectin-3 has been shown to increase invasiveness of human breast cancer cells (59). Galectin-3 was reported to be a chemotactic factor for HUVEC (82), and breast cancer cells (59). Introduction of human galectin-3 cDNA into human breast cancer cells BT-549 that are galectin-3 null and non-tumorigenic in nude mice resulted in the establishment of four galectin-3 expressing clones, three of which acquired tumorigenicity when injected into nude mice (83).

Galectin-3 was found to inhibit apoptosis in a variety of settings after it was first reported that transfection of human Jurkat T cells with galectin-3cDNA conferred resistance to apoptosis induced by anti-Fas antibody and staurosporine (126). In breast cancer cells the expression of galectin-3 inhibited cisplatin-induced apoptosis, and this anti-apoptotic effect was abrogated by substitution of alanine for glycine in the NWGR motif that is expressed by galectin-3 and conserved in the Bcl-2 gene family (3). Recently galectin-3 has been shown to protect breast cancer cells from apoptosis induced by gentistein (29), tumor necrosis factor-α, active oxygen species (77), nitric oxide (79), alteration of mitrochondrial membrane potential, and caspase activation (77), A variety of apoptotic stimuli induced translocation of galectin-3 to the perinuclear membrane where it inhibited the release of cytochrome c (129).

The extracellular activity of galectin-3 in mediating cell-cell and cell-matrix adhesion blocked breast cancer cells from apoptosis induced by loss of cell anchorage (anoikis) and induced GI cell cycle arrest (54, 77). Contact of mammary epithelial cells with the extracellular matrix suppressed expression of interleukin-1 beta converting enzyme (ICE, caspase-1) that induces apoptosis through an integrin-dependent pathway (9). Intact galectin-3 appears to play a novel, multi-faceted role in promoting tumorigenicity and metastasis.

Galectin-3 enhances the adaptive immune response by facilitation of antigen presentation and by increasing the proliferation of mature lymphocytes (reviewed in (98)). The expression of anti-sense but not sense phosphorothioated oligonucleotides for galectin-3 significantly blocked proliferation of T cells (53). Galectin-3 also is thought be enhance innate immunity and acute inflammation. Expression of galectin-3 activates mast cells, basophils, monocytes, and neutrophils. Galectin-3 potentiates the lipopolysaccharide-stimulated production of interleukin-1 by monocytes and induces monocyte chemotaxis. Lactose and rabbit anti-galectin-3 serum inhibited the production of IgE by B cells that was stimulated in vitro by polymorphonuclear leukocytes isolated from patients.

N-terminally truncated forms of galectin-3 can block the activity of intact galectin-3 as a tumorigenic protein that-promotes metastasis, and as a pre-inflammatory mediator. As illustrated in FIG. 2, truncated galectin-3 acts at least partly by preventing the homo-and heterotypic aggregation and the adherence of cells to extracellular matrix proteins. Soluble recombinant N-terminally truncated galectin-3 (galectin-3C) should effectively compete with endogenous galectin-3 for carbohydrate binding sites in the nucleus, cytoplasm, extracellular matrix, and in cell-cell adhesions. The non-carbohydrate binding N-terminal domain of galectin-3 promotes multimerization of the protein when bound to carbohydrates, and enables it to cross link cells to the matrix and other cells. Excess administered galectin-3C, in which the N-terminal carbohydrate binding part of the protein has been removed, will occupy binding sites of endogenous galectin-3 and prevent its homophilic cross-linking and other types of protein-protein binding interactions. Galectin-3C itself will have little cross-linking activity.

The safety, plasma half-life, and efficacy of galectin-3C in an orthotopic mouse model of human breast cancer was evaluated. A recent feasibility study of the anticancer activity of recombinant human galectin-3 N-terminally truncated by exhaustive digestion with Clostridium histiolyticum collagenase type VII (Sigma; St. Louis, Mo.), (36, 75) in an orthotopic mouse model of metastatic breast cancer (22) produced promising results. The digestion produced a 143 amino acid residue polypeptide beginning with Gly-108. In treated animals the number of primary tumors that metastasized was less than controls by 3-fold (P=0.031), and the mean primary tumor volume of the treated animals was 61% less than the controls (P=0.009). The results provide compelling evidence that treatment with an N-terminally truncated form of galectin-3 could useful in treatment and prevention of cancer, especially metastatic cancer.

The N-terminally truncated form of galectin-3 would be valuable as a coating for nanoparticles due to its inherent bioactivity. N-terminally truncated forms of galectin-3 would possess anticancer and antiinflammatory bioactivity and yet retain most of the carbohydrate specificity and affinity of the intact galectin-3 as shown in Table 5.

TABLE 5
Relative Carbohydrate Affinities of Galectin-3 and -3C

| Ligand | Galectin-3 | Galectin-3C |
|---|---|---|
| Galβ1→4Glc (Lactose) | 1 | 0.1 |
| Galβ1→4GlcNAc (LacNAc) | 11.3 | |
| Galβ1→3GlcNAc | 6.3 | |
| Galβ1→4(Fucα1→3)GlcNAc | <0.02 | |
| (LeX) | | |
| Galα1→4Gal | <0.03 | |
| Galα1→3GalαOme | 0.2 | |
| (LacNAc)2 | 20* | 54 |
| (LacNAc)5 | 137 | 60.5 |

*Dissociation constant = 1.3 μM. Galectin-3 and 3C had greater affinity for a number of oligosacchardes than other galectin family members (38, 111).

Galectins are expressed in certain cell types and tissues as illustrated by Table 4. Members of the galectin family other than N-terminally truncated galectin-3 have inherent bioactivity that would increase their attractiveness as cell recognition components (reviewed in (35, 62, 102). Further description of the bioactivity of the galectins is provided below.

Both galectin-8 and -9 are tandem-repeat type galectins. Thus, they have two CRDs that differ in structure and specificity. Both the N-terminal CRD and the C terminal CRD of galectin-9 are required for the lectinA to exert eosinophil chemoattraction. The two CRD domains of galectin-8 and -9 were expressed on separate glutathione S-transferase (GST) fusion proteins. The different domains were found to have varying affinities for specific carbohydrates members (38). Thus, by coating nanoparticles with either the N-terminal or Cterminal CRD different specificity for oligosaccharides can be obtained.

Thus, certain cells and tissues could be targeted for biological modulation by coating nanoparticles with antibody to a specific galectin protein. Antibodies can be monoclonal, polyclonal or recombinant. Conveniently, the antibodies can be prepared against the immunogen or portion thereof for example a synthetic peptide based on the sequence, or prepared recombinantly by cloning techniques or the natural gene product and/or portions thereof can be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Spring Harbor, N.Y., 1988 and Borrebaeck, Antibody Engineering—A Practical Guide, W. H. Freeman and Co., 1992. Antibody fragments can also be prepared from antibodies and include Fab, F(ab′)₂, and Fv by methods known to those skilled in the art.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific. Polyclonal antibody can be produced and purified as IgY from eggs laid by chickens immunized with carrier conjugated antigen from chickens. The immunization procedure lasts for over 6 weeks, the eggs are collected within about 2 months and then IgY is purified.

For producing monoclonal antibodies the technique involves hyperimmunization of an appropriate donor, generally a mouse, with the protein immunogen, and isolation of spleenic antibody producing cells. The protein can be conjugated to an immunogenic carrier such as ovalbumin. These cells are fused to a cell having immortality, such as a myeloma cell, to provide a fused~cell hybrid that has immortality and secretes the required antibody. The cells are then cultured, in bulk, and the monoclonal antibodies harvested from the culture media for use. In mice, animals are immunized over 3 months and sera are periodically tested for antibodies. Splenocytes from animals with acceptable sera antibody activity are fused with a myeloma cell line, and the resulting hybridomas are screened with the same assays used to demonstrate antibody performance. Lastly, limiting dilution cloning of the hybridoma cell line is performed to insure monoclonality of the cell line. Antibody is purified and tested.

For producing recombinant antibody (see (71); (72); Mernaugh and Mernaugh, 1995), messenger RNAs from antibody producing B-lymphocytes of animals, or hybridoma are reverse-transcribed to obtain complimentary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

Certain embodiments of coatings, components, and/or targets include natural and synthetic, native and modified, anionic or acidic saccharides, disaccharides, oligosaccharides, polysaccharides and glycosaminoglycans (GAGs). Dermatan sulfates, for example, have been shown to be useful for targeting molecules specifically to cells, e.g., as in U.S. Pat. No. 6,106,866.

Many peptidic fragments of extracellular matrix molecules are known that are bioactive functions, e.g., the tripeptidic integrin-mediated adhesion domain of fibronectin, see also, e.g., U.S. Pat. Nos. 6,074,659 and 5,646,248.

Moreover, other peptidic targeting ligands can be used, e.g., as in U. S. Pat. No. 5,846,561. Also, for example, lung targeting peptides are set forth in U.S. Pat. No. 6,174,867. Also, for example, organ-targeting peptides can be used, as in U.S. Pat. No. 6,232,287. Also, for example, brain-targeting peptides can be used, as in U.S. Pat. No. 6,296,832. Also, for example, heart-targeting peptides can be used, as in U.S. Pat. No. 6,303,5473.

Moreover, nanoparticles can be targeted for uptake by clatharin coated pits, as well as by caveolae, e.g., as in U.S. Pat. Nos. 5,284,646 and 5,554,386, which include carbohydrates for targeting uses.

The nanoparticles can also include bioactive, diagnostic, or visualization agents that are conjugated to a cell recognition component or a cell recognition target. Such agents can be chemically attached to a cell recognition component, or other ligand, to target the therapeutic agents specifically to a cell or tissue. For example, a toxin can be conjugated to tenascin so as to deliver the toxin to a cancer cell. For example, a cell recognition component set forth herein can be conjugated to a bioactive, diagnostic, or visualization agent set forth herein. Conjugation can involve activating a bioactive, diagnostic, or visualization agent and/or the cell recognition component.

The nanoparticles that include a linking molecule include at least two functional groups that are activated and that react with the bioactive, diagnostic, or visualization agent and/or the cell recognition components so that they can be joined together. The bioactive, diagnostic, and/or visualization agents and/or the cell recognition component and/or the linking molecule can be activated.

The linking molecule can include a degradable group that is enzymatically or hydrolytically degradable so as to release the bioactive, diagnostic, or visualization agents. Examples of degradable groups include the polypeptide sequences cleaved by thrombin, plasmin, collagenase, intracellular proteases, and extracellular proteases. Other examples of degradable groups are lactides, caprolactones, and esters.

Chemistries for conjugating bioactive, diagnostic, or visualization agents to cell recognition components, e.g., proteins, peptides, antibodies, growth factors, ligands, and other cell recognition components or cell recognition targets are known to persons of ordinary skill in these arts, e.g., as in “Chemistry of Protein Conjugation and Cross-Linking” by Shan S. Wong, CRC Press; (Jun. 18, 1991) and Bioconjugate Techniques, Greg T. Hermanson, Academic Press, 1996, San Diego; and in U.S. Pat. No. 6,153,729 (especially with regard to polypeptides).

Moreover, the cell recognition component can be associated with delivery vehicles for delivering the therapeutic, diagnostic, or visualization agent. Examples of delivery vehicles include, e.g., liposomes, DNA particles, nanoparticles, stealth liposomes, polyethylene glycols, macromolecules, gels, hydrogels, controlled release matrices, sponges, degradable scaffolds, and microsponges.

The nanoparticles and particles can include bioactive agents that are delivered -to cells and act to modulate cellular activity. To modulate cellular activity means to increase or decrease some aspect of cellular function, e.g., to increase or decrease synthesis of a protein or action of an enzyme. Bioactive agents or other agents can be delivered for many purposes. Agents can include drugs, proteins, small molecules, toxins, hormones, enzymes, nucleic acids, peptides, steroids, growth factors, modulators of enzyme activity, modulators of receptor activity and vitamins. By directing the agent towards the target where efficacy is to be obtained, and away from other areas where toxicity is obtained, particular cells and tissues can be targeted for research, scientific, and medical purposes. A tissue is a material made by the body, and can include extracellular matrix, structural proteins, and connective tissue. Tissues do not necessarily, but often do, contain cells.

Growth factors are an example of a type of bioactive agent that can be delivered to a cell. As are discussed, growth factors are implicated in many cellular activities, particularly cell proliferation and differentiation. Thus growth factors can be used to modulate many cell activities, including hyperproliferation, differentiation, wound healing, bone formation, and other activities that are regulated by growth factors. Moreover, active moieties of growth factors e.g., polypeptides, are also known.

Small toxins are a type of agent that can be loaded into a nanoparticle and delivered to a cell or tissue. Many small toxins are known to those skilled in the metal parts, including toxins for use in treating cancer. For example, nanoparticles can be loaded with small molecule toxins, including anthracyclines, doxorubicin, vincristine, cyclophosphamide, topotecan, taxol, and paclitaxel. These small toxins are, in general, predominantly hydrophobic and have relatively low MWs, about 1000 or less. Moreover, peptidic oncoagents can also be included.

Further, compounds and agents that have been shown to be useful for modulating cellular activities for a therapeutic or diagnostic use are contemplated. For example, PCT WO 02/100343 describes the use of galectin for hyperproliferative disorders.

The nanoparticles and particles can also include agents that modulate apoptosis, for example, by reducing or increasing the incidence of apoptosis. Apoptosis is a form of programmed cell death that occurs through the activation of cell-intrinsic suicide machinery. Apoptosis plays a major role during development and homeostasis. Apoptosis can be triggered in a variety of cell types by the deprivation of growth factors, which appear to repress an active suicide response. An apoptotic cell breaks apart into fragments of many apoptotic bodies that are rapidly phagocytosed. Inducing apoptosis in cancer cells can be an effective therapeutic approach. Inducing apoptosis in tissue cultured cells provides a model system for studying the effects of certain drugs for triggering, reversing, or halting the apoptotic pathway. Accordingly, increasing a cell's potential to enter the apoptotic pathway, or otherwise modulating apoptosis, is useful.

In vivo administration of oligonucleotide containing nanoparticles can be subdermal, transdermal, subcutaneous, or intramuscular. Intravenous administration or use of implanted pumps can also be used. Doses are selected to provide effective inhibition of cancer cell growth and/or proliferation.

Specifically, some factors for modulating apoptosis include factors that activate or deactivate death receptors, including ligands for death receptors or factors that competitively inhibit the finding of factors to death receptors. Thus there are many factors that are modulators of apoptosis, i.e., that serve to enhance, inhibit, trigger, initiate, or otherwise affect apoptosis. Apoptosis can be triggered by administration of apoptotic factors, including synthetic and natural factors. Some natural factors interact with cell surface receptors referred to death receptors and contribute to, or cause, apoptosis. Death receptors belong to the tumor necrosis factor (TNF) gene superfamily and generally can have several functions other than initiating apoptosis. The best characterized of the death receptors are CD95 (or Fas), TNFRl (TNF receptor-1) and the TRAIL (TNF-related apoptosis inducing ligand) receptors DR4 and DR5.

The bcl-2 proteins are a family of proteins involved in the response to apoptosis. Some of these proteins (such as bcl-2 and bcl-XL) are anti-apoptotic, while others (such as Bad or Bax) are pro-apoptotic. The sensitivity of cells to apoptotic stimuli can depend on the balance of pro-and anti-apoptotic bcl-2 proteins. Thus some factors for modulating apoptosis or factors that up regulate or down regulate bcl-2 proteins, modulate bcl-2 proteins, competitively inhibit such proteins, specifically behind such proteins, or active fragments thereof. Moreover, delivery of bcl-2 proteins can modulate apoptosis.

Caspases are a family of proteins that are effectors of apoptosis. The caspases exist within the cell as inactive pro-forms or zymogens. The zymogens can be cleaved to form active enzymes following the induction of apoptosis. Induction of apoptosis via death receptors results in the activation of an initiator caspase. These caspases can then activate other caspases in a cascade that leads to degradation of key cellular proteins and apoptosis. Thus some factors for modulating apoptosis are factors that up regulate or down regulate caspases, modulate caspases, competitively inhibit caspases, specifically behind caspases, or active fragments thereof. Moreover, delivery of caspases can modulate apoptosis. About 13 caspases are presently known, and are referred to as caspase-1, caspases-2, etc.

Aside from the ligation of death receptors, there are other mechanisms by which the caspase cascade can be activated. For example, Granzyme B can be delivered into cells and thereby directly activate certain caspases. For example, delivery of cytochrome C can also lead to the activation of certain caspases.

An example of an apoptosis modulating factor is casein-kinase-2 alpha (CK2α). CK2α potentiates apoptosis in a eukaryotic cell. CK20α biological activity can be reduced by administering to the cell an effective amount of an anti-sense stand of DNA, RNA, or siRNA. Thus, nanoparticles can be used to potentiate apoptosis in eukaryotic cells by decreasing the expression of CK2α. Apoptosis is inhibited or substantially decreased by preventing transcription of CK-2 DNA and/or translation of RNA. This can be carried out by introducing antisense oligonucleotides of the CK-2 sequence into cells, in which they hybridize to the CK-2 encoding mRNA sequences, preventing their further processing. It is contemplated that the antisense oligonucleotide can be introduced into the cells by introducing antisense-single stranded nucleic acid that is substantially identical to the complement of the cDNA sequence. It is also possible to inhibit expression of CK-2 by the addition of agents that degrade CK-2. Such agents include a protease or other substance that enhances CK-2 breakdown in cells. In either case, the effect is indirect, in that less CK-2 is available than would otherwise be the case.

As used herein, the term nucleic acid refers to both RNA and DNA, including cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand).

Polynucleic acids, such as the sequences set forth herein and fragments thereof, can be used in diagnostics, therapeutics, prophylaxis, and as research reagents and in kits. Provision of means for detecting hybridization of oligonucleotide with a gene, mRNA, or polypeptide can routinely be accomplished. Such provision can include enzyme conjugation, radiolabeling or any other suitable detection systems. Research purposes are also available, e.g., specific hybridization exhibited by the polynucleotides or polynucleic acids can be used for assays, purifications, cellular product preparations and in other methodologies which can be appreciated by persons of ordinary skill in the art.

Polynucleotides are nucleic acid molecules of at least three nucleotide subunits. A nucleotide, as the term is used herein, has three components: an organic base (e.g., adenine, cytosine, guanine, thymine, , or uracil, herein referred to as A, C, G, T, and U, respectively), a phosphate group, and a five-carbon sugar that links the phosphate group and the organic base. In a polynucleotide, the organic bases of the nucleotide subunits determine the sequence of the polynucleotide and allow for interaction with a second polynucleotide. The nucleotide subunits of a polynucleotide are linked by phosphodiester bonds such that the five-carbon sugar of one nucleotide forms an ester bond with the phosphate of an adjacent nucleotide, and the resulting sugar-phosphates form the backbone of the polynucleotide. Polynucleotides described herein can be produced through the well-known and routinely used technique of solid phase synthesis. Similarly, a polynucleotide has a sequence of at least three nucleic acids and can be synthesized using commonly known techniques.

Polynucleotides and polynucleotide analogues (e.g., morpholinos) can be designed to hybridize to a target nucleic acid molecule. The term hybridization, as used herein, means hydrogen bonding, which can be Watson-Crick, Hoogsteen, or reversed Hoogsteen hydrogen bonding, between complementary nucleoside or nucleotide bases. For example, A and T, and G and C, respectively, are complementary bases that pair through the formation of hydrogen bonds. Complementary, as used herein, refers to the capacity for precise pairing between two nucleotides. A nonspecific adsorption or interaction is not considered to be hybridization. For example, if a nucleotide at a certain position of a polynucleotide analogue is capable of hydrogen bonding with a nucleotide at the same position of a target nucleic acid molecule, then the polynucleotide analogue and the target nucleic acid molecule are considered to be complementary to each other at that position. A polynucleotide or polynucleotide analogue and a target nucleic acid molecule are complementary to each other when a sufficient number of corresponding positions in each molecule are occupied by nucleotides that can hydrogen bond with each other. It is understood in the art that the sequence of the polynucleotide or polynucleotide analogue need not be 100% complementary to that of the target nucleic acid molecule to hybridize.

The nanoparticles of the present invention can include various polypeptide sequences and/or purified polypeptides. A polypeptide refers to a chain of amino acid residues, regardless of post-translational modification (e.g., phosphorylation or glycosylation) and/or complexation with additional polypeptides, synthesis into multisubunit complexes, with nucleic acids and/or carbohydrates, or other molecules. Proteoglycans therefore also are referred to herein as polypeptides. A functional polypeptide is a polypeptide that is capable of promoting the indicated function. Polypeptides can be produced by a number of methods, many of which are well known in the art.

The term purified as used herein with reference to a polypeptide refers to a polypeptide that either has no naturally occurring counterpart (e.g., a peptidomimetic), or has been chemically synthesized and is thus substantially uncontaminated by other polypeptides, or has been separated or purified from other most cellular components by which it is naturally accompanied (e.g., other cellular proteins, polynucleotides, or cellular components). An example of a purified polypeptide is one that is at least 70%, by dry weight, free from the proteins and naturally occurring organic molecules with which it naturally associates. A preparation of the a purified polypeptide therefore can be, for example, at least 80%, at least 90%, or at least 99%, by dry weight, the polypeptide. Polypeptides also can be engineered to contain a tag sequence (e.g., a polyhistidine tag, a myc tag) that facilitates the polypeptide to be purified or marked (e.g., captured onto an affinity matrix, visualized under a microscope).

Nucleic acids can be incorporated into vectors. As used herein, a vector is a replicon, such as a plasmid, phage, or cosmid, into which another nucleic acid segment can be inserted so as to bring about replication of the inserted segment. Vectors of the invention typically are expression vectors containing an inserted nucleic acid segment that is operably linked to expression control sequences. An expression vector is a vector that includes one or more expression control sequences, and an expression control sequence is a DNA sequence that controls and regulates the transcription and/or translation of another DNA sequence. Expression control sequences include, for example, promoter sequences, transcriptional enhancer elements, and any other nucleic acid elements required for RNA polymerase binding, initiation, or termination of transcription. With respect to expression control sequences, “operably linked” means that the expression control sequence and the inserted nucleic acid sequence of interest are positioned such that the inserted sequence is transcribed (e.g., when the vector is introduced into a host cell). For example, a DNA sequence is operably linked to an expression-control sequence, such as a promoter when the expression control sequence controls and regulates the transcription and translation of that DNA sequence. The term “operably linked” includes having an appropriate start signal (e.g., ATG) in front of the DNA sequence to be expressed and maintaining the correct reading frame to permit expression of the DNA sequence under the control of the expression control sequence to yield production of the desired protein product. Examples of vectors include, for example, plasmids, adenovirus, Adeno-Associated Virus (AAV), Lentivirus (FIV), Retrovirus (MoMLV), and transposons, e.g., as set forth in U.S. Pat. No. 6,489,458.

There are a variety of promoters that could be used including, e.g., constitutive promoters, tissue-specific promoters, inducible promoters, and the like. Promoters are regulatory signals that bind RNA polymerase in a cell to initiate transcription of a downstream (3′ direction) coding sequence.

Anti-sense DNA compounds (e.g., oligonucleotides) treat disease, and more generally later biological activity, by interrupting cellular production of a target protein. Such compounds offer the potential benefits of 1) rational drug design rather than screening huge compound libraries and 2) a decrease in anticipated side effects due to the specificity of Watson-Crick base-pairing between the antisense molecule's sequential pattern of nucleotide bases and that of the target protein's precursor mRNA. One antisense therapeutic, Vitravene, has been approved for human use in the treatment of AIDS-related CMV retinitis. This drug is applied by intravitreol injection, which aids in maintaining drug concentration due to the isolation of the eye compartment from the systemic circulation.

A polynucleic acid or polynucleic acid analogue can be complementary to a sense or an antisense target nucleic acid molecule. When complementary to a sense nucleic acid molecule, the polynucleic acid is said to be antisense. Thus the identification as sense or antisense is referenced to a particular reference nucleic acid. For example, a polynucleotide analogue can be antisense to an mRNA molecule or sense to the DNA molecule from which an mRNA is transcribed. As used herein, the term “coding region” refers to the portion of a nucleic acid molecule encoding an RNA molecule that is translated into protein. A polynucleotide or polynucleotide analogue can be complementary to the coding region of an mRNA molecule or the region corresponding to the coding region on the antisense DNA strand. Alternatively, a polynucleotide or polynucleotide analogue can be complementary to the non-coding region of a nucleic acid molecule. A non-coding region can be, for example, upstream of a transcriptional start site or downstream of a transcriptional end-point in a DNA molecule. A non-coding region also can be upstream of the translational start codon or downstream of the stop codon in an mRNA molecule. Furthermore, a polynucleotide or polynucleotide analogue can be complementary to both coding and non-coding regions of a target nucleic acid molecule. For example, a polynucleotide analogue can be complementary to a region that includes a portion of the 5′ untranslated region (5′-UTR) leading up to the start codon, the start codon, and coding sequences immediately following the start codon of a target nucleic acid molecule.

Various antisense molecules are set forth herein. The antisense molecules can be preferably targeted to hybridize to the start codon of a mRNA and to codons on either side of the start codon, e.g., within 1-20 bases of the start codon. Other codons, however, can be targeted with success, e.g., any set of codons in a sequence. The procedure for identifying additional antisense molecules is know to those of ordinary skill after reading this disclosure. One procedure is to test antisense molecules of about 20 nucleic acids in a screening assay. Each proposed antisense molecule is tested to determine its effectiveness, and the most promising candidates would form the basis for optimization.

Hybridization of antisense oligonucleotides with mRNA interferes with one or more of the normal functions of mRNA, e.g., translocation of the RNA to a site of protein translation, translation of protein from the RNA, splicing of the RNA to yield one or more mRNA species, and catalytic activity which can be engaged in by the RNA. Binding of specific protein(s) to the RNA can also be interfered with by antisense oligonucleotide hybridization to the RNA.

The function of a gene can be disrupted by delivery of anti-sense DNA or RNA that prevents transcription or translation of the protein encoded by the gene. This can be accomplished by providing an appropriate length oligonucleotide that is complimentary to at least a portion of the messenger RNA (mRNA) transcribed from the gene. The antisense strand hybridizes with the mRNA and targets mRNA destruction by preventing ribosomal translation, and subsequent protein synthesis. The specificity of antisense oligonucleotides arises from the formation of Watson-Crick base pairing between the heterocyclic bases of the oligonucleotide and complimentary bases on the target nucleic acid. Oligonucleotides of greater length (15-30 bases) are preferred because they are more specific, and are less likely to induce toxic complications that might result from unwanted hybridization.

The incorporation of small interfering RNA (SiRNA) molecules, which are double stranded RNA molecules that are capable of mimicking an RNA virus infection. One advantage of using SiRNA molecules is that such molecules are very easy to design. In fact, SiRNA molecules can be based on any portion of a messenger RNA molecule or transcript and still be effective in delivering a therapeutic effect in a target cell. As an example, the casein kinase 2 mRNA transcript can be used to prepare an SiRNA molecule. Furthermore, SiRNA molecules typically have little, if any, binding issues since the SiRNA molecule need not bind to specific portion of the gene in order to be effective.

An example of a system for delivering antisense molecules is a collection of nanoparticles of less than about 200 nm loaded with CK2 and optionally made with tenascin or other cell-specific targeting molecules. Other antisense molecules, including those directed against subunits of CK2, can alternatively be used.

Nanoparticles of less than about 50 nm made with hydrophilic surfactants and the extracellular matrix protein tenascin selectively deliver nucleic acid cargo to solid tumors. This selective uptake is mediated by caveolar endocytosis. Nanoparticle entry into solid tumors is from the surrounding tissue (peritumoral infiltration). Local delivery via peritumoral infiltration can offer advantages over current delivery methods into solid tumors. Further increases in drug efficacy are expected to be obtained by incorporating formats exhibiting higher binding affinities for the target Protein Kinase CK2 mRNA.

Polynucleotide analogues or polynucleic acids are chemically modified polynucleotides or polynucleic acids. Polynucleotide analogues can be generated by replacing portions of the sugar-phosphate backbone of a polynucleotide with alternative functional groups. Morpholino-modified polynucleotides, referred to herein as “morpholinos,” are polynucleotide analogues in which the bases are linked by a morpholino-phosphorodiamidate backbone (See U.S. Pat. Nos. 5,142,047 and 5,185,444).

In addition to morpholinos, other examples of polynucleotide analogues include analogues in which the bases are linked by a polyvinyl backbone peptide nucleic acids (PNAs) in which the bases are linked by amide bonds formed by pseudopeptide 2-aminoethyl-glycine, analogues in which the nucleoside subunits are linked by methylphosphonate groups, analogues in which the phosphate residues linking nucleoside subunits are replaced by phosphoroamidate groups, and phosphorothioated DNAs, analogues containing sugar moieties that have 2′ O-methyl groups (Cook P. D., Antisense Medicinal Chemistry, 1998, Springer, N.Y., pp. 51-101).

Polynucleic acids and polynucleic acid analogue embodiments can be useful for research and diagnostics, and for therapeutic use. Modified nucleic acids are known and can be used with embodiments described herein, for example as described in Antisense Research and Application (Springer-Verlag, Berlin, 1998), and especially as described in the chapter by S. T. Crooke: Chapter 1: Basic Principles of Antisense Therapeutics pp. 1-50; and in Chapter 2 by P. D. Cook: Antisense Medicinal Chemistry pp. 51-101. Some modified backbones for nucleic acid molecules are, for example, morpholinos, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Galectins are useful as bioactive molecules to be delivered by nanoparticles. For example, there is provided an N-terminally truncated galectin-3 lacking the N-terminal 107 amino acids that are produced by enzymatic cleavage of the recombinant protein followed by collection of the pharmacologically active fragment, in the preferred method, and that carries PEG. Alternative methods can also be used to N-terminally truncate galectin-3, such methods are known to those of skill in the art.

Also, there are provided N-terminally truncated human galectin-3 molecules that differ slightly in length. The truncated galectin-3 molecules can be longer or shorter than the 143 amino acid residue N-terminally truncated galectin-3 that is lacking the N-terminal 107 amino acids, but have essentially the same ability to inhibit the carbohydrate binding and multimerization of galectin-3 and, therefore, treat diseases and conditions involving cancer, inflammation, and undesirable immune reactions, including various infections, aging, atherosclerosis, autoimmunity, and other similar diseases or conditions. The N-terminally truncated human galectin-3 molecules that differ slightly in length also can be derivatized with polyethylene glycol for slow-release. The molecules are administered to a patient in need of treatment.

There are provided N-terminally truncated galectin-3 molecules that contain fusion tags on the C-or N-terminus such as the commercially available His-Tag® (Novagen, Madison, Wis.) for pET vector constructs that can contain 6, 8, or 10 histidine residues. These vectors can be used with more than a dozen different fusion tags that possess a variety of unique binding qualities and endoprotease sites allowing for high yields and a variety of ways that can be used to increase the purity of recombinant protein. The N-terminal tags can be cleaved off using enterokinase from Novagen, Roche (Indianapolis, Ind.) or Sigma (St. Louis, Mo.). A C-terminal histidine₆ tag can be used for immobilized metal-affinity chromatography, is not immunogeneic, does not alter the affinity of most proteins, and has been used in both preclinical animal and human clinical trials (25). The methods to produce these slightly different versions of the N-terminally truncated human galectin-3 protein are known to those of skill in the art.

A plasmid containing the complete galectin-3 coding sequence is used as a template in a PCR reaction using primers designed to amplify the desired fragment.

Forward primer:
5′ GACGACGACAAGGGCGCCCCTGCTGGG 3′
Reverse primer:
5′ GAGGAGAAGCCCGGTTTATATCATGGTATA 3′

Underlined sequences in each of the primers match the plasmid sequences for pET32 (EK/LIC expression system, Novagen, Madison, Wis.). The reverse primer defines the C-terminal protein sequence and does not differ in these procedures. The noncapitalized portion of the forward primer defines the N-truncated version of the native galectin-3 that, for example begins with Gly-108 (“delta 1-107”, starting at amino acid sequence glycine, alanine, proline, alanine, etc.). The capitalized sequences are added as tails and used to fuse the PCR, product with pET32 Ek/LIC plasmid using the Ek/LIC ligation protocol (Novagen, Madison, Wis.). This plasmid produces a fusion protein with a variety of unique binding qualities and endoprotease sites allowing for high yields and purity of the recombinant protein. It should be noted that more than one cysteine can be introduced to the construct by simply including more cysteine codons (either tgt or tgc) to create a version of N-truncated galectin-3 with one or more cysteines at the N-terminus.

The amino acid sequence of the N-terminally truncated recombinant human galectin-3 that is produced by exhaustive digestion with collagenase, and that can be produced by other cloning methods is designated as SEQ ID NO. 1, and is as follows:

gap agplivpynl pipggvvprm litilgtvkp nanrialdfq rgndvafhfn prfnennrrv ivcntkldnn wgreerqsvf pfesgkpfki qvlvepdhfk vavndahllq ynhrvkkine isklgisgdi dltsasytmi

The amino acid sequence of the N-terminally truncated recombinant human galectin-3 produced by cloning methods to contain one additional cysteine on the N-terminus is designated as SEQ ID NO. 2, and is as follows:

cgap agplivpynl plpggvvprm litilgtvkp nanrialdfq rgndvafhfn prfnennrrv ivcntkldnn wgreerqsvf pfesgkpfki qvlvepdhfk vavndahllq ynhrvkklne isklgisgdi dltsasytmi

The amino acid sequence of the intact recombinant human galectin-3 described by Oda et al (46) is designated as SEQ ID NO. 3, and its sequence is as follows:

1 madnfslhda Isgsgnpnpq gwpgawgnqp agaggypgas ypgaypgqap pgaypgqapp

61 gayhgapgay pgapapgvyp gppsgpgayp ssgqpsapga ypatgpygap agplivpynl

121 plpggvvprm litilgtvkp nanrialdfq rgndvafhfn prfnennrrv ivcntkldnn

181 wgreerqsvf pfesgkpfki qvivepdhfk vavndahllq ynhrvkklne isklgisgdi

241 ditsasytmi

More than two decades have passed since the first descriptions of the effect of the covalent attachment PEG on the circulating life and immunogenicity of proteins (1). A PEGylated version of interferon-alpha by Schering-Plough Corporation was approved by the F.D.A. for use in the U.S. for chronic hepatitis C. Administration of the PEGylated version of interferon-alpha is subcutaneous injection once weekly rather than three times weekly.

Many methods and PEG derivatives are available for conjugation of proteins to PEG. PEG modification of antibodies to tumors has been found to enhance penetration into the tumors and to increase the antitumor effects (45). The circulating lives of single chain antibodies have been extended by conjugated with circulating lives of single chain antibodies have been extended by conjugated with PEG to the carboxylic acid groups or the primary amines. An increase in the polymer length rather than total mass was found to be more effective for serum half-life extension (61).

Specific PEG derivatives react with thiols such as the amino acid residue cysteine (120). These include PEG-ortopyridyl-disulphide, PEG-malemide, and PEG-vinylsulphone. In a preferred method, N-terminally truncated human galectin-3 that is lacking the 107 amino acids on the N-terminus or is similar in size is derivatized on the single cysteine in the sequence (SEQ ID NO. 1). In an alternate method, the N-terminally truncated human galectin-3 is produced with one or more cysteine residues on the N-terminus as described above (SEQ ID NO. 2). Then one or more of the cysteine residues is derivatized with a thiol reactive PEG derivative. Employing a branched PEG derivative and performing the derivatization in the presence of ligand can prevent active site residue derivatization due to steric hindrance (120).

One example of the use in vivo of N-terminally truncated galectin-3 is as a potential therapeutic agent for breast cancer. It was determined that therapy with an N-terminally truncated form of galectin-3 is efficacious for inhibition of metastases. To this end, recombinant galectin-3 was produced and the N-terminally truncated galectin-3 was derived by collagenase enzyme digestion and affinity chromatography. Injected N-terminally truncated galectin-3 was detected by metabolic labeling with ³⁵S methionine prior to collagenase cleavage. The maximum tolerated dose of N-terminally truncated galectin-3 in nude mice was determined to be greater than 125 mg/kg without overt adverse effects. The pharmacokinetic elimination half-life of N-terminally truncated galectin-3 administered intramuscularly into nude mice was found to be 4.56 hours. Mice bearing orthotopically implanted tumors derived from breast cancer cell line MDA-MB435 were treated intramuscularly twice daily for 90 days with N-terminally truncated galectin-3 or a vehicle control. It was found that the mean tumor volumes and weights were statistically significantly less in mice treated with N-terminally truncated galectin-3 compared with control mice, and that fewer numbers of mice exhibited lymph node metastases in the treated group compared with the control group. In a recent study at the end of the treatment period, the number of treated animals with primary tumors that metastasized was less than controls by 3-fold (P=0.031), and the mean primary tumor volume of the treated animals was 61% less than the controls (P=0.009). The results provide compelling evidence that treatment with an N-terminally truncated form of galectin-3 could useful in treatment and prevention of cancer, especially metastatic cancer. The truncated form of galectin-3 was efficacious in reducing metastases, and the tumor volumes and weights of primary tumors.

The methodology for assessing carbohydrate binding of proteins such as an N-terminally truncated galectin-3 is well known to the skilled artisan. Specifically, various laboratories have examined the portion of galectin-3 that is involved in carbohydrate recognition including among these Seetharaman et al., (8), who disclosed the detailed X-ray crystal structure of the carbohydrate recognition domain complexed with N-acetyllactosamine. In addition, the minimal lactose-binding domain of galectin-3 has been extensively mapped by use of a bacteriophage □ surface expression vector (80). These detail the portions of SEQ ID, NO. 2 that are required for carbohydrate binding. This information can be used to define the minimal portion of SEQ ID NO. 3 that are required for binding to carbohydrates and that are thereby required for inclusion in an N-terminally truncated galectin-3 for treatment of cancer that acts by inhibiting the functionality of the wild-type galectin-3.

Accordingly, various studies have examined the portion of galectin-3 that is required for homodimerization, cell cross-linking, hemagglutination, or homotypic aggregation when the galectin-3 is bound to a carbohydrate ligand. These studies include those by Massa et al. (75), Hsu, et al. (41), Gong et al. (32), and Ochieng et al. (86). Massa et al. showed that the carbohydrate binding fragment of recombinant human galectin-3 that was produced by exhaustive digestion with collagenase type VII from Clostridium histiolyticum was unable to increase cooperativity in binding of the intact galectin-3 as the intact galectin-3 protein was able to do (75). In their study Hsu et al. demonstrated that the product of digestion by collagenase enzyme from Achromobacter iophagus (Boehringer Mannheim) cleaved intact human galectin-3 site between Ala-111 and Gly-112. The C-terminal fragment that retained carbohydrate binding failed to hemagglutinate rabbit erythrocytes, an activity possessed by wild type galectin-3 (41). Similarly, Ochieng et al. show that a different fragment of intact galectin-3 produced by metalloproteinases that also contains the carbohydrate recognition domain and retains the ability to bind to carbohydrates fails to homodimerize or hemagglutinate at concentrations at which the wild-type galectin-3 does homodimerize and hemagglutinate (86).

The human metalloproteinase enzymes MMP-2 and MMP-9 can cleave wild type galectin-3 between Ala-62 and Tyr-63 producing two fragments, one containing part of the N-terminal domain and the other containing the carbohydrate recognition domain (85). Gong et al. define another N-terminally truncated galectin-3 that has lost hemagglutination activity and that does not induce tumorigenicity unlike wild-type galectin-3. The N-terminal 11 amino acid residues (following the N-terminal Met residue from the initiation codon) were deleted in the N-terminally truncated galectin-3 described by Gong et al. (32). In a subsequent study Yoshii et al. (128) demonstrated that galectin-3 proteins with point mutations at Ser-6 failed to protect cells from programmed cell death induced by loss of cell anchorage. Cell anchorage also can be described as the cross linking of cells with cells that leads to hemagglutination or cross linking cells with the extracellular matrix.

The exact sequences of the set of the polypeptides based on SEQ. ID NO. 3 that would bind to carbohydrates such as lactose and that would not form homodimers are defined studies such as those cited above. The work shows that N-terminally truncated galectin-3 polypeptides that possess these physical characteristics can be used to treat cancer by reducing tumorigenicity and metastasis. Thus, by correlating function with activity it is possible to further define the present invention in terms of a method of treating cancer using polypeptide fragments of the structure shown in SEQ ID NO 3. This method of treating cancer specifically uses any of a set of polypeptides that include an amino acid sequence of SEQ ID NO 3 that begins with any of the amino acid residues from Leu-7 through Arg-129, and that extends at least as far as any of the amino acid residues from Asp-241 through Ile-250.

The methodology to produce and isolate any one of the specific polypeptides is well-known to the skilled artisan. This could be achieved starting with a plasmid containing the complete galectin-3 coding sequence as a template in a PCR reaction using primers designed to amplify the specific fragment desired. Using the primers designed to amplify the specific desired portion of SEQ ID NO. 3, the PCR product is fused with an expression plasmid such as that for the pET32 Ek/LIC plasmid using the Ek/LIC ligation protocol (Novagen) as described in the specification in reference to the production of SEQ ID NO 1. Thus, a skilled artisan can produce these polypeptides by adapting the method described in the specification to the specific polypeptide desired or by other methods that are well known to those skilled in the art.

Also provided are other members of the galectin family that have inherent bioactivity that would make it therapeutically beneficial to deliver them in nanoparticles (reviewed in (35, 62, 102). Galectin-1 has been found to be beneficial in the animal model of a number of diseases including myasthenia gravis, multiple sclerosis, and rheumatoid arthritis. In a model of nephritis, treatment with galectin-1, galectin-3, or galectin-9 ameliorated disease (116). Both galectin-1 and -9 can induce apoptosis in T cells whereas galectin-3 can protect cells from apoptosis. Thus, expression of galectin-1 or galectin-9 on the cell surface could enhance tumors by suppressing anti-tumor immunity via the apoptotic effect on antigen activated T cells. Expression of galectin-7 in keratinocytes is induced by the tumor suppressor p53 and is associated with cell death due to UVB (7, 74). Expression of galectin-12 in adipocytes induced apoptosis (40).

Both galectin-8 and -9 are tandem-repeat type galectins. Thus, they have two CRDs that differ in structure and specificity. Both the N-terminal CRD and the C-terminal CRD of galectin-9 are required for the lectin to exert eosinophil chemoattraction. The two CRD domains of galectin-8 and -9 were expressed on separate glutathione S-transferase (GST) fusion proteins. The different domains were found to have varying affinities for specific carbohydrates members (38). By using the galectin-8N, or -8C alone it can be possible to block eosinophil chemoattraction. In a similar manner it can be beneficial to use the galectin-9N or 9C alone without the other CRD.

Galectin-8 expression is reported to be decreased in some types of cancers (17), but fingerprinting a panel of 61 human brain, colon, lung, and breast tumor cell lines by polymerase chain reaction (PCR) revealed widespread expression of both galectin-3 and galectin-8 (58). Galectin-8 modulates cell adhesion (65) and has been reported to induce apoptosis (33). The other galectins that can induce apoptosis include galectin-1, -9, -7, and -12 (reviewed in [Rabinovich, 2002 #1923. Unlike the other members of the galectin family recent studies show that the mRNA for the galectin-8 gene encode for six different isoforms of the protein (8).

The present invention provides a method and composition for treating diseases and conditions involving hyperproliferative diseases such as cancer, inflammation, and undesirable immune reactions, various infections, aging, atherosclerosis, autoimmunity, and other diseases by administering nanoparticles including an effective amount of a bioactive molecule that is a “galectin”. The “galectin” can be a galectin protein or nucleic acids including DNA, RNA, mRNA encoding a galectin protein, and including antisense RNA or DNA to galectin nucleic acid, or an antibody that specifically binds to a galectin protein or an antibody that specifically binds to a carbohydrate ligand of a galectin. The galectin proteins can be prepared as a form of recombinant protein in Escherichia coli BL21 (DE3) under the control of the T7 promoter as either intact forms or fusion forms as described previously (38). Expression plasmids for the intact proteins can be constructed using pET expression systems (Novagen). Coding regions can be amplified by PCR using forward primers containing an Ndel site and reverse primers containing a termination codon, and original cDNA plasmids as templates. The amplified fragments can be cloned in pCRII vector (Invitrogen) by a TA cloning strategy according to the manufacturer's instruction. The inserts can be cut out with relevant restriction enzymes, and ligated into an appropriate pET vector. The E. coli BL21, (DE3) can be transformed with the constructed expression plasmids, the nucleotide sequences of which are confirmed. Production of galectin protein can be induced by the addition of 1 mM isopropyl-□-D-thiogalactoside. Soluble factors can be obtained for subsequent purification by affinity chromatography.

The bioactive molecules delivered by nanoparticles in the present invention can be utilized in a combination therapy. This can include adding to the pharmaceutically acceptable carrier additional anti-inflammatory, immunosuppressive compounds, or other similar compositions.

Nanoparticles can include antibodies for targeting the nanoparticles to cells or tissues, whereby bioactive or visualization agents associated with the nanoparticles can be delivered. These can include antibodies having specific binding activity for a cell recognition target, e.g., cell surface receptor, extracellular matrix molecule, growth factor receptor, or cell specific marker. Such antibodies can be useful for directing nanoparticles to specific cell types, for example. The term antibody or antibodies includes intact molecules as well as fragments thereof that are capable of binding to an epitope. The term “epitope” refers to an antigenic determinant on an antigen to which an antibody binds. The terms antibody and antibodies include polyclonal antibodies, monoclonal antibodies, humanized or chimeric antibodies, single chain Fv antibody fragments, Fab fragments, and F(ab)₂ fragments.

Antibodies can be generated according to methods known to those skilled in these arts, e.g., recombinantly, or via hybridoma processes. Further, monoclonal antibodies can be obtained by any technique that provides for the production of antibody molecules by, for example, continuous cell lines in culture as described by Kohler et al. (1975) Nature 256:495-497; the human B-cell hybridoma technique of Kosbor et al. (1983) Immunology Today 4:72 and Cote et al. (1983) Proc. Natl. Acad. Sci. USA 80:2026-2030; and the EBV-hybridoma technique of Cole et al. Monoclonal Antibodies and Cancer Therapy, Alan R. Liss, Inc. pp. 77-96 (1983). Such antibodies can be of any immunoglobulin class, including IgM, IgG, IgE, IgA, IgD, and any subclass thereof. A hybridoma producing the monoclonal antibodies of the invention can be cultivated in vivo or in vivo. A chimeric antibody can be a molecule in which different portions are derived from different animal species, such as those having a variable region derived from a mouse monoclonal antibody and a human immunoglobulin constant region. Chimeric antibodies can be produced through standard techniques.

A monoclonal antibody also can be obtained by using commercially available kits that aid in preparing and screening antibody phage display libraries. An antibody phage display library is a library of recombinant combinatorial immunoglobulin molecules. Examples of kits that can be used to prepare and screen antibody phage display libraries include the Recombinant Phage Antibody System (Pharmacia, Peapack, N.J.) and SurfZAP Phage Display Kit (Stratagene, La Jolla, Calif.). Once produced, antibodies or fragments thereof can be tested for recognition of a polypeptide by standard immunoassay methods including, for example, enzyme-linked immunosorbent assay (ELISA) or radioimmuno assay (RIA).

One method of targeting a cell or tissue is to deliver nanoparticles, e.g., nanocapsules, directly to a location at or near the cell or tissue, e.g., by use of a needle, catheter, transcutaneous delivery system, or suppository.

Nanoparticles penetrate tissues and are able to reach cells for which they are targeted. Thus s50 nanoparticles comprising ligands that are targeted to certain cell types can preferentially interact with the targeted cells instead of other cells. Nanoparticles can be targeted to a cell and be expected to interact specifically with that cell. When nanoparticles comprising tenascin were targeted to cells that preferentially express the tenascin receptor,-the uptake of the nanoparticles was inhibited by the presence of free tenascin. This result shows that the tenascin s50 nanoparticles interacted with the cells using a mechanism that specifically involved tenascin. Thus other cells can be targeted using s50 nanoparticles that have factors that are specific for targets on those cells and can be expected to be preferentially taken up by those cells.

Typical sizes for nanoparticles containing plasmid DNA can be in the range of 10 to 25 nm of dry diameter. Such particles are useful when extracellular delivery of a particle cargo is desired. Some example of such uses would include, for example, delivery of particle cargo on the outside of a cell, especially for delivery of peptides, proteins, sugars and small molecules.

Nanoparticles can be targeted to cancerous cells and to cells involved in other hyperproliferative disorders, with the nanoparticles having bioactive, diagnostic, and/or visualization agents. Several experimental treatments for recurrent cancer, e.g., SCCHN, are in later clinical trials or near market approval. They include, for example, INGN 201 (p53 replacement gene therapy delivered by adenovirus), intratumoral Onyx-015 (mutant adenovirus that replicates in p53-/-cells combined with cisplatin/5-FU) and Erbitux (IMCL C 225, humanized antibody to the EGR receptor). These treatments, however, could all benefit from a better method of delivery e.g., via nanoparticles.

Hyperproliferative disorders can involve genes that ultimately affect gene transcription through their interaction with the DNA scaffold, e.g., histones and chromatin structures. For example, the involvement of nuclear receptors in cancer is documented by mutations in the retinoic acid receptor (RAR), found in acute promyelocytic leukemia (APL), hepatocellular carcinomas and lung cancer. Such alterations can lead to the deregulated recruitment of enzymes having histone deacetylase (HDAC) activity to cause alteration of gene expression. Inhibition of HDACs could thus block gene transcriptional activity and result cellular differentiation of tumor cells, subsequently preventing the cells from further growth or even induce cell death, see also U.S. Pat. Ser. No. 60/428,296, filed Nov. 22, 2002.

Also provided are methods for using probes to detect protein, receptor, or ligand expression in a cell preparation, cell, tissue, or tissue sample. For example, a technique such as in situ hybridization with a nanoparticle directed against a particular cell surface receptor can be used to detect the cell surface molecule in a tissue on a slide (e.g., a tumor tissue). Such probes can be labeled with a variety of markers, including radioactive, chemiluminescent, and fluorescent markers, for example. Alternatively, an immunohistochemistry technique with an anti-protein antibody conjugated to a nanoparticle can be used to detect the protein in a cell or a tissue.

Cells and/or tissues can be specifically targeted for many purposes, including for therapeutic, diagnostic, research, and labeling purposes. As already discussed, nanoparticles are described herein that are configured to enter cells via caveolae, a mechanism for cell entry that has many advantages compared to other entry mechanisms. Moreover, such nanoparticles are so small that they penetrate the spaces between cells and move freely through tissues. Indeed, nanoparticles of less than about 70 or 50 nm in diameter are much smaller than the spaces between cells. For example, suitably sized nanoparticles can pass out of blood vessels through the spaces between endothelial cells that line the blood vessels, and into the vascular media. Thus intravascular delivery of suitably sized nanoparticles allows for the nanoparticles to be delivered to tissues beyond the vasculature.

In general, the range of possible targets can be dependent on the route of administration e.g. intravenous or intra-arterial, subcutaneous, intra-peritoneal, intrathecal, intracranial, bronchial, and so forth. For systemic injections, the specificity of this delivery system is affected by the accessibility of the target to blood borne particles, which in turn, is affected by the size range of the particles.

Particles with size less than 150 nanometers can access the interstitial space by traversing through the fenestrations that line most blood vessel walls. Under such circumstances, the range of cells that can be targeted is extensive. Some non-exhaustive examples of cells that can be targeted includes the parenchymal cells of the liver sinusoids, the fibroblasts of the connective tissues, myofibroblasts, epidermal cells, dermal cells, cells exposed by injury, the cells in the Islets of Langerhans in the pancreas, cardiac myocytes, chief and parietal cells of the intestine, osteocytes and chrondocytes in the bone, chondrocytes in cartilage, keratinocytes, nerve cells of the peripheral nervous system, epithelial cells of the kidney and lung, Sertoli cells of the testis, and so forth.

For subcutaneous injections, the targetable cells include all cells that reside in the connective tissue (e.g., fibroblasts, mast cells, etc.), Langerhans cells, keratinocytes, and muscle cells. For intrathecal injections, the targetable cells include neurons, glial cells, astrocytes, and blood-brain barrier endothelial cells. For intraperitoneal injection, the targetable cells include the macrophages and neutrophil. Active endothelial transport has been demonstrated for small molecules (transcytosis). Transendothelial migration of macromolecular conjugates and noncovalent paired-ion formulations of drugs and diagnostic agents with sulfated glycosaminoglycan, having a combined size of between about 8000 daltons and about 500 nm are accelerated by the infusion of sulfated glycosaminoglycans (i.e. dermatan sulfate) which become selectively bound to the induced endothelial receptors at sites of disease.

Many aspects of particle delivery are described herein. Delivery of a particle can entail delivery of the particle itself or delivery of the particle as well as structures or compounds that the particle is attached to or associated with. After reading this disclosure, a person of ordinary skill will understand how to adapt methods for using particles that exceed the size for caveolar delivery to the delivery of nanoparticles for caveolar delivery, and how such techniques can used for delivery of larger particles to extracellular sites, tissue, and the like. Delivery techniques used for delivery of particles can, in general, be adapted to use with nanoparticles.

The particles can be delivered by any known method adapted to the application. Examples of delivery of a particle include via injection, including intravenously, intramuscularly, or subcutaneously, and in a pharmaceutically acceptable solution and sterile vehicles, such as physiological buffers (e.g., saline solution or glucose serum). The particle can also be administered orally or rectally, when they are combined with pharmaceutically acceptable solid or liquid excipients. Particles can also be administered externally, for example, in the form of an aerosol with a suitable vehicle suitable for this mode of administration, for example, nasally. Further, delivery through a catheter or other surgical tubing is possible. Alternative routes include tablets, capsules, and the like, nebulizers for liquid formulations, and inhalers for lyophilized or aerosolized ligands.

Presently known methods for delivering molecules in vivo and in vivo, including small molecules or peptides, can be used for particles. Such methods include use with microspheres, liposomes, other microparticle vehicles or controlled release formulations placed in certain tissues, including blood. Examples of controlled release carriers include semipermeable polymer matrices in the form of shaped articles, e.g., suppositories, or microcapsules. A variety of suitable delivery methods are set forth in, for example, U.S. Pat. Nos. 5,626,877; 5,891,108; 5,972,027; 6,041,252; 6,071,305, 6,074,673; 6,083,996; 6,086,582; 6,086,912; 6,110,498; 6,136,295; 6,142,939; 6,235,313; 6,245,349; 6,251,079; 6,283,947; 6,283,949; 6,287,792; 6,309,375; 6,309,380; 6,309,410; 6,317,629; 6,346,272; 6,350,780; 6,379,382; 6,387,124; 6,387,397 6,416,778 and 6,296,832.

Also contemplated are pharmaceutical compositions and formulations that include a collection of particles or molecules embodied herein. Pharmaceutical compositions containing nanoparticles can be applied topically (e.g., to surgical incisions or diabetic skin ulcers). Formulations for topical administration of nanoparticles include, for example, sterile and non-sterile aqueous solutions, non-aqueous solutions in common solvents such as alcohols, or solutions in liquid or solid oil bases. Such solutions also can contain buffers, diluents and other suitable additives. Formulations for topical administration can include transdermal patches, ointments, lotions, creams, gels, drops, suppositories, sprays, liquids, and powders. Coated prophylactics, gloves and the like also can be useful. Conventional pharmaceutical carriers, aqueous, powder or oily bases, thickeners and the like can be necessary or desirable. Alternatively, pharmaceutical compositions containing nanoparticles can be administered orally or by injection (e.g., by subcutaneous, intradermal, intraperitoneal, or intravenous injection).

For oligonucleotides, examples of pharmaceutically acceptable salts include, e.g., (a) salts formed with cations such as sodium, potassium, ammonium, etc.; (b) acid addition salts formed with inorganic acids, for example, hydrochloric acid, hydrobromic acid (c) salts formed with organic acids e.g., for example, acetic acid, oxalic acid, tartaric acid; and (d) salts formed from elemental anions e.g., chlorine, bromine, and iodine.

In general, for any substance, a pharmaceutically acceptable carrier is: a material that is combined with the substance for delivery to an animal. Conventional pharmaceutical carriers, aqueous, powder or oily bases, thickeners and the like can be necessary or desirable. In some cases the carrier is essential for delivery, e.g., to solubilize an insoluble compound for liquid delivery; a buffer for control of the pH of the substance to preserve its activity; or a diluent to prevent loss of the substance in the storage vessel. In other cases, however, the carrier is for convenience, e.g., a liquid for more convenient administration. Pharmaceutically acceptable carriers are used, in general, with a compound so as to make the compound useful for a therapy or as a product.

Nanoparticles can be frozen or reconstituted for later use or can be delivered to a target cell or tissue by such routes of administration as oral, intravenous, subcutaneous, intraperitoneal, intrathecal, intramuscular, inhalational, topical, transdermal, suppository (rectal), pessary (vaginal), intra urethral, intraportal, intrahepatic, intra-arterial, intra-ocular, transtympanic, intratumoral, intrathecal, transmucosal, buccal, or any combination of any of these.

In another application, the nanoparticles can be designed for specific cellular or tissue uptake by polymer selection and/or inclusion of cell-recognition components in a nanoparticle biocompatible polymer shell or coating. Such coatings will have utility for specific or increased delivery of the bioactive agent to the target cell. Alternatively, instead of coating, the cell recognition components can be a component of the nanoparticles. Such applications include, e.g., tumor-targeting of the chemotherapeutic agents or anti-sense DNA, antigen delivery to antigen-presenting cells, ocular delivery of ribozymes to retinal cells, transdermal delivery of protein antibodies, or transtympanic membrane delivery of peptide nucleic acids.

Additional embodiments include peritumoral infiltration techniques, e.g., as described in U.S. Pat. No. 5,945,100. Increased penetration and/or reduced backflow and diversion through the point of entry can be achieved to enhance delivery to a tumor using peritumoral infiltration so that more material is introduced into and remains in the tumor. Such infiltration can be achieve, for example, through the use of a viscous vehicle, most preferably one having a similar density to tissue, for the material to be delivered. Preferred materials include solutions or suspensions of a polymeric material that gel or solidify at the time of or shortly after injection or implantation into or near the tumor. In an embodiment, the solution is injected via a catheter or needle into or near the regions of the tumor to be treated.

Additionally, the present invention provides a method and composition for treating diseases and conditions involving inflammation, and undesirable immune reactions, including various infections, aging, atherosclerosis, and autoimmunity by administering nanoparticles including an effective amount of a galectin formulated for sustained or slow-release and placed in a pharmaceutically acceptable carrier. The present invention also provides a method and composition for treating diseases and conditions involving inflammation, and undesirable immune reactions, including various infections, aging, atherosclerosis, and autoimmunity by administering an effective amount of antibody that specifically binds to carbohydrate ligands of galectin-3.

Additionally, the compounds of the present invention can be utilized in a combination therapy. This can include adding to the pharmaceutically acceptable carrier additional anti-inflammatory, immunosuppressive compounds, or other similar compositions.

The present invention also provides liposomes (70-100 nm) of 1-palmitoyl-2-oleoylphosphatidylcholine, cholesterol, and poly(ethylene glycol) (PEG)-modified phosphatidylethanolamine (PEG-DSPE) or immunoliposomes that are also conjugated to Fab′ fragments of a humanized recombinant MAb against one or more cell recognition targets such as the extracellular domain of HER2/neu to create sterically stabilized immunoliposomes (anti-HER2 SL) delivering at least one galectin. Alternatively, the present invention also provides liposomes conjugated to a least one galectin, or conjugated to Fab′ fragments of a humanized recombinant MAb that binds to a galectin, as a cell recognition component. Conjugation employed maleimide-terminated membrane-anchored spacers of two kinds: a short spacer, providing attachment of Fab′ close to the liposome bilayer, or a long spacer, with Fab′ attachment at the distal terminus of the PEG chain. The present invention provides for liposomes that optimize internalization of a drug into target cells bearing a characteristic cell recognition target. The liposomes comprise a cell recognition component that is q galectin, an amphipathic vesicle-forming lipid, and a polyethylene glycol derivatized lipid, and a biologically active molecule. The immunoliposomes comprise a cell recognition component that is a Fab′ domain of an antibody that specifically binds a galectin or another characteristic marker, an amphipathic vesicle-forming lipid, and a polyethylene glycol derivatized lipid, and a biologically active molecule. Certain embodiments are described in the following Examples, which are intended as illustrations only, since numerous modifications and variations will be apparent to those skilled in the art after reading this disclosure.

### EXAMPLES

### Example 1

### Ligand-Based Nanoparticles for Enhanced Delivery of Anti-Tumor Compounds, Particularly Antisense Compounds to the Casein Kinase 2 Molecule

Cisplatin was nanoencapsulated into the various candidate tumor-binding agents as described previously and nanoparticles were compared for growth inhibition in a metastatic variant of Alva-41 prostate carcinoma cells and Ca-9-22. Formulas were tested in duplicate in two separate experiments. Results are illustrated for the prostate cell line in FIG. 3. Referring to FIG. 3, PEX-MMP-l/Cisplatin refers to s50 nanoparticles comprising cisplatin and the Recombinant Pex binding domain of membrane-associated Matrix Metalloproteinase-1 (see Bello et. al, Cancer Research (2001) 61: 8730-36); Tenascin/Cisplatin refers to s50 nanoparticles having tenascin and cisplatin, FN-PHSCN/Cisplatin refers to nanoparticles comprising the FN-PHSCN fragment and cisplatin, galectin-3/cisplatin refers to s50 nanoparticles comprising recombinant N-terminally truncated galectin-3 and cisplatin, hyaluronan/cisplatin refers to s50 nanoparticles comprising hyaluronan and cisplatin, and naked cisplatin refers to the addition of free cisplatin to the cell medium. In these experiments cells were plated at 5,000 per well and followed for 72 hours. IC₅₀'s for growth inhibition ranged from 60 μM to 200 μM for the nanoencapsulated cisplatins compared to 100 μM for free cisplatin. As a comparison, based on a standard male patient, an acceptable in vitro dose of cisplatin would correspond to about 10 μg/ml or 30 μM. Given the reasonable expectation of a 10 to 100-fold increase in maximum tolerated dose by targeted delivery, any of these particles could reasonably be considered for additional pharmaceutical development. This data shows that numerous types of molecules, regardless of their structure but, with consideration of their role in cell pathobiology, can be usefully nanoencapsulated in multiple appropriate components to exhibit broad anti-tumor activity.

Free cisplatin and the nanocapsules comprising hyaluronan or N-terminally truncated galectin-3 showed the greatest activity at doses greater than 60 □g/ml compared to the other nanocapsule formulations. Hyaluronan is a large glycosaminoglycan, and the mass of the recombinant hyaluronan used in this study was 1 million kD. Advantages of recombinant N-terminally truncated galectin-3 is that it is not glycosylated, ranges in size from about 15 to 35. kD, and can be easily purified using affinity chromatography from cultures of genetically modified Escherichia colibacteria. Bacteria do not glycosylate proteins, thus, hyaluronan must be isolated from mammalian cells or sources.

Other advantages of galectins as cell targeting components include the following: (a) enhanced cellular uptake via the inherent property of the galectins for stimulation of their own endocytosis into the cytoplasm or into the cytoplasm and nucleus of some cells, and trancystosis and shuttling from one cell into another (18, 23, 44, 66, 67); (b) the inherent bioactivity of specific galectins that would be beneficial in cancer, inflammatory states, and other diseases; (c) and, the ability to target nanocapsules to specific tissues or cells based on varying affinity of galectins for specific glycoconjugates.

### Example 2

### Effectiveness of Nanoencapsulated Compounds Against Tumor Nests in Organ Culture

To confirm the in vitro biological activity of nanoencapsulated anti-tumor compounds, 3 formulations were tested against 3-D in vivo tumor nests grown in pig dermis organ culture, see FIG. 4. The three compounds were nanoparticles comprising tenascin and phosphodiester antisense CK2α (118); nanoparticles comprising recombinant N-terminally truncated galectin-3 and casein kinase 2 alpha (CK2□) phosphodiester antisense and nanoparticles comprising hyaluronan and cisplatin. Porcine skin biopsies (8 mm diameter), were either injected or not with carcinoma cells and cultured in duplicate at an air-water interface on a 300 μm stainless steel mesh in commercially available organ culture dishes. At 0.5 to 3 days post injection, biopsies were treated topically with nanoencapsulated phosphodiester antisense to CK2□, a small molecule anti-tumor agent or buffer, then organ-cultured for 3 days. Tumor-bearing biopsies were snap frozen in liquid nitrogen, then cryosectioned into 6 micron sections for tumor detection using immunofluorescence microscopy. Tumors were detected by either immunosignal for keratin 14 (K-14, SSCHN), prostate-specific antigen (psa, prostate carcinoma), or apoptosis via the TUNEL method. Descriptive results are summarized in FIG. 4 and the following Table 6.

TABLE 6
Efficacy of nanoencapsulated compounds in model of
minimum residual disease.

| | | | | | |
|---|---|---|---|---|---|
| | | | | Time | |
| | Cells | Time lag | | lag | |
| | injected | from | | from | |
| | into | tumor | Tumor | injec- | |
| Tumor nest | porcine | injection | nest | tion to | |
| (dose/molecule/ | skin | to | starting | treat- | Tumor nest description |
| particle) | biopsy | treatment | description | ment | at termination |

TUMOR: SSCHN Ca-9-22 psg. 28, p6F1

| | | | | | |
|---|---|---|---|---|---|
| 0 μg | 200,000 | NA | NA | 5 | Primary tumor along |
| | | | | days | injection, scattered |
| | | | | | nests throughout biopsy |
| 2 μg antisense | 200,000 | 18 hours | NA | 5 | none |
| TN | | | | days | |

TUMNOR: SSCHN SCC-15, psg. 4, p26F1

| | | | | | |
|---|---|---|---|---|---|
| 0 μg | 200,000 | NA | mm, | 8 | Primary tumor along |
| | | | CK2-(+), | days | injection, diffuse cell |
| | | | K-14-(+) | | groups throughout |
| | | | □v□6-(+) | | biopsy, complete |
| | | | □v□3-(+) | | colonization of |
| | | | | | epidermis |
| 0.5 μg antisense | 200,000 | 3 days | | 8 | 400 μm primary tumor |
| TN | | | | days | nest, epidermis |
| 1 μg antisense | 200,000 | 3 days | | 8 | Still present epidermis |
| TN | | | | days | |
| 2 μg sense TN | 200,000 | 3 days | | 8 | Possibly increased |
| | | | | days | epidermal colonization, |
| | | | | | □v□6-(+) and apoptotic |
| | | | | | by TUNL |
| 2 μg antisense | 200,000 | 3 days | | 8 | No tumor cells by K-14 |
| TN | | | | days | detection |

TUMOR: Prostate Carcinoma Alva-41, psg. 371, p33F3

| | | | | | |
|---|---|---|---|---|---|
| 0 μg | 200 | 3 days - | 50 μm nest | 5 | Biopsy was dead - a |
| | | couldn't | plus | days | problem with tumor |
| | | find | primary | | overgrowth |
| | | injection | | | |
| | | site | | | |
| 5 μg antisense | 200 | | | 5 | Biopsy alive, no tumor |
| truncated | | | | days | by PSA at injection site |
| recombinant | | | | | |
| galectin 3 (rtG3) | | | | | |
| 50 μg antisense | 200 | | | 5 | Biopsy alive, no tumor |
| rtG3 | | | | days | |
| 5 μg cisplatin HA | 200 | | | 5 | Biopsy was dead, few |
| | | | | days | scattered living |
| | | | | | carcinoma cells |
| 50 μg cisplatin | 200 | | | 5 | Biopsy alive, but |
| HA | | | | days | epidermis appears |
| | | | | | PSA-(+). |

From these results it can be concluded that nanoencapsulated compounds, especially antisense, showed excellent anti-tumor activity in a reasonable model of minimum residual disease. Minimum residual disease refers to small nests of tumor left behind following surgical removal of the primary tumor or in the bloodstream following chemotherapy, but has not recruited an independent blood supply. As shown in Table 8 above, the cisplatin-loaded nanocapsules with recombinant N-terminally truncated galectin-3 were more efficacious than the capsules with hyaluronan. At a dose of 5 □g the cisplatin-loaded nanocapsules with recombinant 10 N-terminally truncated galectin-3had better anti-tumor activity than the capsules with hyaluronan at a dose of 50 □g.

### Example 3

### Inhibition of Metastasis and Tumorigeneicity in Orthotopic Nude Mouse Model of Human Breast Cancer by Antisense Galectin-3C Nanocapsules

N-terminally truncated galectin-3 nanoparticles containing antisense are prepared for functional growth inhibition studies by dispersion atomization using the 20-mer phosphodiester sequence spanning the translation start site of the alpha subdomain of casein kinase 2 alpha (CK2a) (PO, 11207p, Pepperkok, 1991). In brief, s50-nanoparticles are produced by: a) dispersing 200 μg of antisense DNA oligonucleotide complexed with 60 mcg of 15K MW polyornithine into sterile water using a water-insoluble surfactant system of 8 μg of TM-diol in 50% DMSO; b) emulsifying the dispersed nucleic acid by sonication with a water-miscible solvent, 150 μl of DMSO; c) inverting emulsion with 750 μl of PBS addition; d) “coating” hydrophobic micelles by ligand mixture addition, 10 μg of 16 kD N-terminally truncated galectin-3 and adsorption; and e) atomizing ligand-stabilized micelles into a salt receiving solution (200 mM Li⁺, 10 mM Ca²⁺). Following overnight incubation, particles are collected by centrifugation from the mother liquor for decanting and 0.2 μM filter sterilization. Cisplatin containing nanocapsules coated with N-terminally truncated galectin-3 are prepared as described (118). Encapsulation yield is measured at 74% using a standard overnight protein K digestion at 56° C. followed by isobutanol extraction and recovery of DNA on an anionic column. Average particle size is less than 50 nm as measured by tapping mode atomic force microscopy of a 0.1 μg/ml sample dried down on a mica sheet.

Female athymic CD-1 nude mice between 4 and 5 weeks of age are bred and maintained in a HEPA-filtered environment with cages, food and bedding sterilized by autoclaving. The breeding pairs are obtained from the Charles River Laboratories (Wilmington, Mass.). The animal diets are obtained from Harlan Teklad (Madison, Wis.). Ampicillin (Sigma, St. Louis, Mo.) at a concentration of 5% (v/v) is added to the autoclaved drinking water. Breast cancer cell line MDA-MB-435, that expresses galectin-3, is transfected with a plasmid expressing green fluorescent protein (GFP), and cells are injected into the subcutis of nude mice to form solid tumors. Anesthetized test animals are transplanted by surgical orthotopic implantation using fragments harvested from the subcutaneously growing tumors. An incision approximately 0.5 cm long is made on the second right mammary gland and two fragments of 1 mm³ of MDA-MB-435-GFP tumor tissue are sutured into the fat pad of the gland with a sterile nylon 8-0 surgical suture. All surgical and animal manipulations and procedures are conducted under HEPA-filtered laminar flow hoods. The orthotopically-transplanted animals used for the study are selected to establish groups of similar mean tumor size and body weight. Groups for each of the cohort conditions are randomly chosen. Administration of the treatments is begun when tumors reached palpable sizes. There are three groups of animals, one vehicle only control group and one group treated with antisense nanocapsules (10 mg/kg/day treated topically by applying sequential 50 μl aliquots for 5 minutes each for 3 days). One group is treated with antisense nanocapsules as above with one dose of 3 mg/kg cisplatin nanocapsules coated with N-terminally truncated galectin-3 injected into the tumor.

The primary tumors are measured with a pair of calipers daily after initiation of treatment through the end of the study, as well as by computer imaging of the relative diameters of the fluorescent tumors. Parameters determined at the end of are volume of primary tumor based on caliper measurements and calculated as (W²×length/2), number of metastases based on tumor histology, and animal weight. Whole body imaging of the GFP expression by the MDA-MB-435 is carried out in a light box illuminated by blue-light fiber optics (Lightools Research, Encinitas, Calif.) and imaged using a thermoelectrically cooled C5810 3-chip Hamamatsu CCD camera (Hamamatsu Photonic Systems, Bridgewater N.J.) as previously described.

At autopsy, tissue samples from the axillary lymph node, the liver and the lungs are collected, fixed in 10% formalin, embedded in paraffin and sectioned, and then processed through standard procedures of hematoxylin and eosin staining for subsequent microscopic examination.

Throughout this application various publications are referenced. Full citations for the publications are listed below. The disclosures of these publications in their entireties are hereby incorporated by reference into this application to more fully describe the state of the art to which this invention pertains. The invention has been described in an illustrative manner, and it is to be understood that the terminology that has been used is intended to be in the nature of words of description rather than of limitation. Obviously, many modifications and variations of the present invention are possible in light of the above teachings. It is, therefore, to be understood that within the scope of the appended claims, the invention can be practiced otherwise than as specifically described.

### REFERENCES

1. Abuchowski, A., J. R. McCoy, N. C. Palczuk, T. van Es, and F. F. Davis. 1977. Effect of covalent attachment of polyethylene glycol on immunogenicity and circulating life of bovine liver catalase. J Biol Chem 252:3582-6.

2. Ackerman, S. J., L. Liu, M. A. Kwatia, M. P. Savage, D. D. Leonidas, G. J. Swaminathan, and K. R. Acharya. 2002. Charcot-Leyden crystal protein (galectin-10) is not a dual function galectin with lysophospholipase activity but binds a lysophospholipase inhibitor in a novel structural fashion. J Biol Chem 277:14859-68.

3. Akahani, S., P. Nangia-Makker, H. Inohara, H. R. Kim, and A. Raz. 1997. Galectin-3: a novel antiapoptotic molecule with a functional BH1 (NWGR) domain of Bcl-2 family. Cancer Res 57:5272-6.

4. Almkvist, J., J. Faldt, C. Dahlgren, H. Leffler, and A. Karlsson. 2001. Lipopolysaccharide-induced gelatinase granule mobilization primes neutrophils for activation by galectin-3 and formylmethionyl-Leu-Phe. Infect Immun 69:832-7.

5. Baum, L. G., M. Pang, N. L. Perillo, T. Wu, A. Delegeane, C. H. Uittenbogaart, M. Fukuda, and J. J. Seilhamer. 1995. Human thymic epithelial cells express an endogenous lectin, galectin-1, which binds to core 2 O-glycans on thymocytes and T lymphoblastoid cells. J Exp Med 181:877-87.

6. Bello, L., V. Lucini, G. Carrabba, C. Giussani, M. Machluf, M. Pluderi, D. Nikas, J. Zhang, G. Tomei, R. M. Villani, R. S. Carroll, A. Bikfalvi, and P. M. Black. 2001. Simultaneous inhibition of glioma angiogenesis, cell proliferation, and invasion by a naturally occurring fragment of human metalloproteinase-2. Cancer Res 61:8730-6.

7. Bernerd, F., A. Sarasin, and T. Magnaldo. 1999. Galectin-7 overexpression is associated with the apoptotic process in UVB-induced sunburn keratinocytes. Proc Natl Acad Sci U S A 96:11329-34.

8. Bidon, N., F. Brichory, P. Bourguet, J. P. Le Pennec, and L. Dazord. 2001. Galectin-8: a complex sub-family of galectins (Review). Int J Mol Med 8:245-50.

9. Boudreau, N., C. J. Sympson, Z. Werb, and M. J. Bissell. 1995. Suppression of ICE and apoptosis in mammary epithelial cells by extracellular matrix. Science 267:891-3.

10. Bourne, Y., B. Bolgiano, D. I. Liao, G. Strecker, P. Cantau, O. Herzberg, T. Feizi, and C. Cambillau. 1994. Crosslinking of mammalian lectin (galectin-1) by complex biantennary saccharides. Nat Struct Biol 1:863-70.

11. Brewer, F. C. 2002. Binding and cross-linking properties of galectins. Biochim Biophys Acta 1572:255.

12. Castronovo, V., F. A. Van Den Brule, P. Jackers, N. Clausse, F. T. Liu, C. Gillet, and M. E. Sobel. 1996. Decreased expression of galectin-3 is associated with progression of human breast cancer. J Pathol 179:43-8.

13. Chammas, R., M. G. Jasiulionis, A. M. Ventura, L. R. Travassos, and R. R. Brentani. 1996. Laminin-binding proteins in EJ-ras-transformed fibroblasts. Braz J Med Biol Res 29:1141-9.

14. Chen, J. W., N. Madamanchi, N. R. Madamanchi, T. T. Trier, and M. J. Keherly. 2001. Lamp-1 is upregulated in human glioblastoma cell lines induced to undergo apoptosis. J Biomed Sci 8:365-74.

15. Craig, S. S., P. Krishnaswamy, A. M. Irani, C. L. Kepley, F. T. Liu, and L. B. Schwartz. 1995. Immunoelectron microscopic localization of galectin-3, an IgE binding protein, in human mast cells and basophils. Anat Rec 242:211-9.

16. Danguy, A., I. Camby, and R. Kiss. 2002. Galectins and cancer. Biochim Biophys Acta 1572:285.

17. Danguy, A., S. Rorive, C. Decaestecker, Y. Bronckart, H. Kaltner, Y. R. Hadari, R. Goren, Y. Zich, M. Petein, I. Salmon, H. J. Gabius, and R. Kiss. 2001. Immunohistochemical profile of galectin-8 expression in benign and malignant tumors of epithelial, mesenchymatous and adipous origins, and of the nervous system. Histol Histopathol 16:861-8.

18. Davidson, P. J., M. J. Davis, R. J. Patterson, M. A. Ripoche, F. Poirier, and J. L. Wang. 2002. Shuttling of galectin-3 between the nucleus and cytoplasm. Glycobiology 12:329-37.

19. Demetriou, M., M. Granovsky, S. Quaggin, and J. W. Dennis. 2001. Negative regulation of T-cell activation and autoimmunity by Mgat5 N-glycosylation. Nature 409:733-9.

20. Dennis, J. W., M. Granovsky, and C. E. Warren. 1999. Glycoprotein glycosylation and cancer progression. Biochim Biophys Acta 1473:21-34.

21. Dunphy, J. L., G. J. Barcham, R. J. Bischof, A. R. Young, A. Nash, and E. N. Meeusen. 2002. Isolation and characterization of a novel eosinophil-specific galectin released into the lungs in response to allergen challenge. J Biol Chem 277:14916-24.

22. Fu, X., P. Le, and R. M. Hoffman. 1993. A metastatic orthotopic-transplant nude-mouse model of human patient breast cancer. Anticancer Res 13:901-4.

23. Furtak, V., F. Hatcher, and J. Ochieng. 2001. Galectin-3 mediates the endocytosis of beta-1 integrins by breast carcinoma cells. Biochem Biophys Res Commun 289:845-50.

24. Furuta, K., M. Ikeda, Y. Nakayama, K. Nakamura, M. Tanaka, N. Hamasaki, M. Himeno, S. R. Hamilton, and J. T. August. 2001. Expression of lysosome-associated membrane proteins in human colorectal neoplasms and inflammatory diseases. Am J Pathol 159:449-55.

25. Gaberc-Porekar, V., and V. Menart. 2001. Perspectives of immobilized-metal affinity chromatography. J. Biochem. Biophys. Methods 49:335-360.

26. Gaudin, J. C., B. Mehul, and R. C. Hughes. 2000. Nuclear localisation of wild type and mutant galectin-3 in transfected cells. Biol Cell 92:49-58.

27. Gitt, M. A., M. F. Wiser, H. Leffler, J. Herrmann, Y. R. Xia, S. M. Massa, D. N. Cooper, A. J. Lusis, and S. H. Barondes. 1995. Sequence and mapping of galectin-5, a beta-galactoside-binding lectin, found in rat erythrocytes. J Biol Chem 270:5032-8.

28. Gitt, M. A., Y. R. Xia, R. E. Atchison, A. J. Lusis, S. H. Barondes, and H. Leffler. 1998. Sequence, structure, and chromosomal mapping of the mouse Lgals6 gene, encoding galectin-6. J Biol Chem 273:2961-70.

29. Glinsky, G. V., J. E. Price, V. V. Glinsky, V. V. Mossine, G. Kiriakova, and J. B. Metcalf. 1996. Inhibition of human breast cancer metastasis in nude mice by synthetic glycoamines. Cancer Res 56:5319-24.

30. Glinsky, V. V., G. V. Glinsky, K. Rittenhouse-Olson, M. E. Huflejt, O. V. Glinskii, S. L. Deutscher, and T. P. Quinn. 2001. The role of Thomsen-Friedenreich antigen in adhesion of human breast and prostate cancer cells to the endothelium. Cancer Res 61:4851-7.

31. Glinsky, V. V., M. E. Huflejt, G. V. Glinsky, S. L. Deutscher, and T. P. Quinn. 2000. Effects of Thomsen-Friedenreich antigen-specific peptide P-30 on beta-galactoside-mediated homotypic aggregation and adhesion to the endothelium of MDA-MB-435 human breast carcinoma cells. Cancer-Res 60:2584-8.

32. Gong, H. C., Y. Honjo, P. Nangia-Makker, V. Hogan, N. Mazurak, R. S. Bresalier, and A. Raz. 1999. The NH2 terminus of galectin-3 governs cellular compartmentalization and functions in cancer cells. Cancer Res 59:6239-45.

33. Hadari, Y. R., R. Arbel-Goren, Y. Levy, A. Amsterdam, R. Alon, R. Zakut, and Y. Zick. 2000. Galectin-8 binding to integrins inhibits cell adhesion and induces apoptosis. J Cell Sci 113:2385-97.

34. Harrison, L. E., J. G. Guillem, P. Paty, and A. M. Cohen. 1997. Preoperative carcinoembryonic antigen predicts outcomes in node-negative colon cancer patients: a multivariate analysis of 572 patients. J Am Coll Surg 185:55-9.

35. Hernandez, J. D., and L. G. Baum. 2002. Ah, sweet mystery of death! Galectins and control of cell fate. Glycobiology 12:127R-36R.

36. Herrmann, J., C. W. Turck, R. E. Atchison, M. E. Huflejt, L. Poulter, M. A. Gitt, A. L. Burlingame, S. H. Barondes, and H. Leffler. 1993. Primary structure of the soluble lactose binding lectin L-29 from rat and dog and interaction of its non-collagenous proline-, glycine-, tyrosine-rich sequence with bacterial and tissue collagenase. J Biol Chem 268:26704-11.

37. Hikita, C., S. Vijayakumar, J. Takito, H. Erdjument-Bromage, P. Tempst, and Q. Al-Awqati. 2000. Induction of terminal differentiation in epithelial cells requires polymerization of hensin by galectin 3. J Cell Biol 151:1235-46.

38. Hirabayashi, J., T. Hashidate, Y. Arata, N. Nishi, T. Nakamura, M. Hirashima, T. Urashima, T. Oka, M. Futai, W. Muller, F. Yagi, and K. Kasai. 2002. Oligosaccharide specificity of galectins: a search by frontal affinity chromatography. Biochim Biophys Acta 1572:232.

39. Hornberger, L. K., S. Singhroy, T. Cavalle-Garrido, W. Tsang, F. Keeley, and M. Rabinovitch. 2000. Synthesis of extracellular matrix and adhesion through beta(1) integrins are critical for fetal ventricular myocyte proliferation. Circ Res 87:508-15.

40. Hotta, K., T. Funahashi, Y. Matsukawa, M. Takahashi, H. Nishizawa, K. Kishida, M. Matsuda, H. Kuriyama, S. Kihara, T. Nakamura, Y. Tochino, N. L. Bodkin, B. C. Hansen, and Y. Matsuzawa. 2001. Galectin-12, an Adipose-expressed Galectin-like Molecule Possessing Apoptosis-inducing Activity. J Biol Chem 276:34089-97.

41. Hsu, D. K., R. I. Suberi, and F. T. Liu. 1992. Biochemical and biophysical characterization of human recombinant IgE-binding protein, an S-type animal lectin. J. Biol. Chem. 267:14167-14174.

42. Hsu, D. K., R. Y. Yang, Z. Pan, L. Yu, D. R. Salomon, W. P. Fung-Leung, and F. T. Liu. 2000. Targeted disruption of the galectin-3 gene results in attenuated peritoneal inflammatory responses. Am J Pathol 156:1073-83.

43. Hughes, R. C. 2001. Galectins as modulators of cell adhesion. Biochimie 83:667-76.

44. Hughes, R. C. 1999. Secretion of the galectin family of mammalian carbohydrate-binding proteins. Biochim Biophys Acta 1473:172-85.

45. Hurwitz, E., L. N. Klapper, M. Wilchek, Y. Yarden, and M. Sela. 2000. Inhibition of tumor growth by poly(ethylene glycol) derivatives of anti-ErbB2 antibodies. Cancer Immunol Immunother 49:226-34.

46. ldikio, H. 1998. Galectin-3 expression in human breast carcinoma: correlation with cancer histologic grade. Int J Oncol 12:1287-90.

47. Inohara, H., S. Akahani, K. Koths, and A. Raz. 1996. Interactions between galectin-3 and Mac-2-binding protein mediate cell-cell adhesion. Cancer Res 56:4530-4.

48. Inohara, H., Y. Honjo, T. Yoshii, S. Akahani, J. Yoshida, K. Hattori, S. Okamoto, T. Sawada, A. Raz, and T. Kubo. 1999. Expression of galectin-3 in fine-needle aspirates as a diagnostic marker differentiating benign from malignant thyroid neoplasms. Cancer 85:2475-84.

49. Inohara, H., and A. Raz. 1995. Functional evidence that cell surface galectin-3 mediates homotypic cell adhesion. Cancer Res 55:3267-71.

50. Ishihara, A., T. Yoshida, H. Tamaki, and T. Sakakura. 1995. Tenascin expression in cancer cells and stroma of human breast cancer and its prognostic significance. Clin Cancer Res 1:1035-41.

51. Iurisci, l., N. Tinari, C. Natoli, D. Angelucci, E. Cianchetti, and S. lacobelli. 2000. Concentrations of galectin-3 in the sera of normal controls and cancer patients. Clin Cancer Res 6:1389-93.

52. Jacob, K., M. Webber, D. Benayahu, and H. K. Kleinman. 1999. Osteonectin promotes prostate cancer cell migration and invasion: a possible mechanism for metastasis to bone. Cancer Res 59:4453-7.

53. Joo, H. G., P. S. Goedegebuure, N. Sadanaga, M. Nagoshi, W. von Bernstorff, and T. J. Eberlein. 2001. Expression and function of galectin-3, a beta-galactoside-binding protein in activated T lymphocytes. J Leukoc Biol 69:555-64.

54. Kim, H. R., H. M. Lin, H. Biliran, and A. Raz. 1999. Cell cycle arrest and inhibition of anoikis by galectin-3 in human breast epithelial cells. Cancer Res 59:4148-54.

55. Kronenwett, R., S. Martin, and R. Haas. 2000. The role of cytokines and adhesion molecules for mobilization of peripheral blood stem cells. Stem Cells 18:320-30.

56. Kuwabara, I., Y. Kuwabara, R. Y. Yang, M. Schuler, D. R. Green, B. L. Zuraw, D. K. Hsu, and F. T. Liu. 2002. Galectin-7 (PIG1) exhibits pro-apoptotic function through JNK activation and mitochondrial cytochrome c release. J Biol Chem 277:3487-97.

57. Kuwabara, I., and F. T. Liu. 1996. Galectin-3 promotes adhesion of human neutrophils to laminin. J Immunol 156:3939-44.

58. Lahm, H., S. Andre, A. Hoeflich, J. R. Fischer, B. Sordat, H. Kaltner, E. Wolf, and H. J. Gabius. 2001. Comprehensive galectin fingerprinting in a panel of 61 human tumor cell lines by RT-PCR and its implications for diagnostic and therapeutic procedures. J Cancer Res Clin Oncol 127:375-86.

59. Le Marer, N., and R. C. Hughes. 1996. Effects of the carbohydrate-binding protein galectin-3 on the invasiveness of human breast carcinoma cells. J Cell Physiol 168:51-8.

60. Lee, E. C., H. J. Woo, C. A. Korzelius, G. D. Steele, Jr., and A. M. Mercurio. 1991. Carbohydrate-binding protein 35 is the major cell-surface laminin-binding protein in colon carcinoma. Arch Surg 126:1498-502.

61. Lee, L. S., C. Conover, C. Shi, M. Whitlow, and D. Filpula. 1999. Prolonged circulating lives of single-chain Fv proteins conjugated with polyethylene glycol: a comparison of conjugation chemistries and compounds. Bioconjug Chem 10:973-81.

62. Leffler, H. 2001. Galectins structure and function—a synopsis. Results Probl Cell Differ 33:57-83.

63. Lenander, C., J. K. Habermann, A. Ost, B. Nilsson, H. Schimmelpenning, K. Tryggvason, and G. Auer. 2001. Laminin-5 gamma 2 chain expression correlates with unfavorable prognosis in colon carcinomas. Anal Cell Pathol 22:201-9.

64. Leonidas, D. D., E. H. Vatzaki, H. Vorum, J. E. Celis, P. Madsen, and K. R. Acharya. 1998. Structural basis for the recognition of carbohydrates by human galectin-7. Biochemistry 37:13930-40.

65. Levy, Y., R. Arbel-Goren, Y. R. Hadari, S. Eshhar, D. Ronen, E. Elhanany, B. Geiger, and Y. Zick. 2001. Galectin-8 functions as a matricellular modulator of cell adhesion. J Biol Chem 276:31285-95.

66. Lindstedt, R. 1993. University of Lund, Lund, Sweden. 67. Lindstedt, T., G. Apodaca, S. H. Barondes, K. Mostov, and H. Leffler. 1993. Apical secretion nof a cytosolic protein by Madin-Darby canine kidney cells. Evidence for polarized release of an endogenous lectin by a nonclassical secretory pathway. J Biol Chem 268:11750-11757.

68. Liu, F., R. Patterson, and J. Wang. 2002. Intracellular functions of galectins. Biochim Biophys Acta 1572:263.

69. Liu, F. T., D. K. Hsu, R. I. Zuberi, P. N. Hill, A. Shenhav, I. Kuwabara, and S. S. Chen. 1996. Modulation of functional properties of galectin-3 by monoclonal antibodies binding to the non-lectin domains. Biochemistry 35:6073-9.

70. Liu, F. T., D. K. Hsu, R. I. Zuberi, I. Kuwabara, E. Y. Chi, and W. R. Henderson, Jr. 1995. Expression and function of galectin-3, a beta-galactoside-binding lectin, in human monocytes and macrophages. Am J Pathol 147:1016-28.

71. Livant, D. L., R. K. Brabec, K. J. Pienta, D. L. Allen, K. Kurachi, S. Markwart, and A. Upadhyaya. 2000. Anti-invasive, antitumorigenic, and antimetastatic activities of the PHSCN sequence in prostate carcinoma. Cancer Res 60:309-20.

72. Lohi, J. 2001. Laminin-5 in the progression of carcinomas. Int J Cancer 94:763-7.

73. Madsen, P., H. H. Rasmussen, T. Flint, P. Gromov, T. A. Kruse, B. Honore, H. Vorum, and J. E. Celis. 1995. Cloning, expression, and chromosome mapping of human galectin-7. J Biol Chem 270:5823-9.

74. Magnaldo, T., F. Bernerd, and M. Darmon. 1995. Galectin-7, a human 14-kDa S-lectin, specifically expressed in keratinocytes and sensitive to retinoic acid. Dev Biol 168:259-71.

75. Massa, S. M., D. N. Cooper, H. Leffler, and S. H. Barondes. 1993. L-29, an endogenous lectin, binds to glycoconjugate ligands with positive cooperativity. Biochemistry 32:260-7.

76. Matarrese, P., O. Fusco, N. Tinari, C. Natoli, F. T. Liu, M. L. Semeraro, W. Malorni, and S. lacobelli. 2000. Galectin-3 overexpression protects from apoptosis by improving cell adhesion properties. Int J Cancer 85:545-54.

77. Matarrese, P., N. Tinari, M. Semeraro, C. Natoli, S. lacobelli, and W. Malorni. 2000. Galectin-3 overexpression protects from cell damage and death by influencing mitochrondrial homeostatis. FEBS letters 473:311-315.

78. Menon, R. P., and R. C. Hughes. 1999. Determinants in the N-terminal domains of galectin-3 for secretion by a novel pathway circumventing the endoplasmic reticulum-Golgi complex. Eur J Biochem 264:569-76.

79. Moon, B. K., Y. J. Lee, P. Battle, J. M. Jessup, A. Raz, and H. R. Kim. 2001. Galectin-3 protects human breast carcinoma cells against nitric oxide-induced apoptosis: implication of galectin-3 function during metastasis. Am J Pathol 159:1055-60.

80. Moriki, T., I. Kuwabara, F. T. Liu, and 1. N. Maruyama. 1999. Protein domain mapping by lambda phage display: the minimal lactose-binding domain of galectin-3. Biochem Biophys Res Commun 265:291-6.

81. Nakamura, M., H. Inufusa, T. Adachi, M. Aga, M. Kurimoto, Y. Nakatani, T. Wakano, A. Nakajima, J. I. Hida, M. Miyake, K. Shindo, and M. Yasutomi. 1999. Involvement of galectin-3 expression in colorectal cancer progression and metastasis. Int J Oncol 15:143-8.

82. Nangia-Makker, P., Y. Honjo, R. Sarvis, S. Akahani, V. Hogan, K. J. Pienta, and A. Raz. 2000. Galectin-3 induces endothelial cell morphogenesis and angiogenesis. Am J Pathol 156:899-909.

83. Nangia-Makker, P., R. Sarvis, D. W. Visscher, J. Bailey-Penrod, A. Raz, and F. H. Sarkar. 1998. Galectin-3 and Li retrotransposons in human breast carcinomas. Breast Cancer Res Treat 49:171-83.

84. Nies, D. E., T. J. Hemesath, J. H. Kim, J. R. Gulcher, and K. Stefansson. 1991. The complete cDNA sequence of human hexabrachion (Tenascin). A multidomain protein containing unique epidermal growth factor repeats. J Biol Chem 266:2818-23.

85. Ochieng, J., R. Fridman, P. Nangia-Makker, D. E. Kleiner, L. A. Liotta, W. G. Stetler-Stevenson, and A. Raz. 1994. Galectin-3 is a novel substrate for human matrix metalloproteinases-2 and -9. Biochemistry 33:14109-14.

86. Ochieng, J., B. Green, S. Evans, O. James, and P. Warfield. 1998. Modulation of the biological functions of galectin-3 by matrix metalloproteinases. Biochim Biophys Acta 1379:97-106.

87. Ochieng, J., M. L. Leite-Browning, and P. Warfield. 1998. Regulation of cellular adhesion to extracellular matrix proteins by galectin-3. Biochem Biophys Res Commun 246:788-91.

88. Ochieng, J., and P. Warfield. 1995. Galectin-3 binding potentials of mouse tumor EHS and human placental laminins. Biochem,Biophys Res Commun 217:402-6.

89. Ochieng, J., P. Warfield, B. Green-Jarvis, and 1. Fentie. 1999. Galectin-3 regulates the adhesive interaction between breast carcinoma cells and elastin. J Cell Biochem 75:505-14.

90. Oda, Y., H. Leffler, Y. Sakakura, K. Kasai, and S. H. Barondes. 1991. Human breast carcinoma cDNA encoding a galactoside-binding lectin homologous to mouse Mac-2 antigen. Gene 99:279-83.

91. Ogden, A. T., l. Nunes, K. Ko, S. Wu, C. S. Hines, A. F. Wang, R. S. Hegde, and R. A. Lang. 1998. GRIFIN, a novel lens-specific protein related to the galectin family. J Biol Chem 273:28889-96.

92. Ohannesian, D. W., D. Lotan, P. Thomas, J. M. Jessup, M. Fukuda, H. J. Gabius, and R. Lotan. 1995. Carcinoembryonic antigen and other glycoconjugates act as ligands for galectin-3 in human colon carcinoma cells. Cancer Res 55:2191-9.

93. Openo, K. P., M. M. Kadrofske, R. J. Patterson, and J. L. Wang. 2000. Galectin-3 expression and subcellular localization in senescent human fibroblasts. Exp Cell Res 255:278-90.

94. Ordonez, C., R. A. Screaton, C. llantzis, and C. P. Stanners. 2000. Human carcinoembryonic antigen functions as a general inhibitor of anoikis. Cancer Res 60:3419-24.

95. Perillo, N., C. Uittenbogaart, J. Nguyen, and L. Baum. 1997. Galectin-1, an endogenous lectin produced by thymic epithelial cells, induces apoptosis of human thymocytes. J Exp Med 185:1851-1858.

96. Perillo, N. L., M. E. Marcus, and L. G. Baum. 1998. Galectins: versatile modulators of cell adhesion, cell proliferation, and cell death. Journal of Molecular Medicine 76:402-412.

97. Prochiantz, A. 2000. Messenger proteins: homeoproteins, TAT and others. Curr Opin Cell Biol 12:400-6.

98. Rabinovich, G., N. Rubinstein, and M. Toscano. 2002. Role of galectins in inflammatory and immunomodulatory processes. Biochim Biophys Acta 1572:274.

99. Rabinovich, G. A. 1999. Galectins: an evolutionarily conserved family of animal lectins with multifunctional properties; a trip from the gene to clinical therapy. Cell Death Differ 6:711-21.

100. Rabinovich, G. A., L. G. Baum, N. Tinari, R. Paganelli, C. Natoli, F. T. Liu, and S. lacobelli. 2002. Galectins and their ligands: amplifiers, silencers or tuners of the inflammatory response? Trends Immunol 23:313-20.

101. Rabinovich, G. A., C. M. Riera, C. A. Landa, and C. E. Sotocanor. 1999.

Galectins: a key intersection between glycobiology and immunology. Braz J Med Biol Res 32:383-93.

102. Rabinovich, G. A., N. Rubinstein, and L. Fainboim. 2002. Unlocking the secrets of galectins: a challenge at the frontier of glyco-immunology. J Leukoc Biol 71:741-52.

103. Raz, A., D. G. Zhu, V. Hogan, N. Shah, T. Raz, R. Karkash, G. Pazerini, and P. Carmi. 1990. Evidence for the role of 34-kDa galactoside-binding lectin in transformation and metastasis. Int J Cancer 46:871-7.

104. Rousseau, C., M. P. Muriel, M. Musset, J. Botti, and A. P. Seve. 2000. Glycosylated nuclear lectin CBP70 also associated with endoplasmic reticulum and the Golgi apparatus: does the “classic pathway” of glycosylation also apply to nuclear glycoproteins? J Cell Biochem 78:638-49.

105. Saitoh, O., W. C. Wang, R. Lotan, and M. Fukuda. 1992. Differential glycosylation and cell surface expression of lysosomal membrane glycoproteins in sublines of a human colon cancer exhibiting distinct metastatic potentials. J Biol Chem 267:5700-11.

106. Sarafian, V., M. Jadot, J. M. Foidart, J. J. Letesson, F. Van den Brule, V. Castronovo, R. Wattiaux, and S. W. Coninck. 1998. Expression of Lamp-1 and Lamp-2 and their interactions with galectin-3 in human tumor cells. Int J Cancer 75:105-11.

107. Sato, S., and R. C. Hughes. 1992. Binding specificity of a baby hamster kidney lectin for H type I and II chains, polylactoamine glycans and appropriately glycosylated forms of laminin and fibronectin. J Biol Chem 267: 983-6990.

108. Schoeppner, H. L., A. Raz, S. B. Ho, and R. S. Bresalier. 1995. Expression of an endogenous galactose-binding lectin correlates with neoplastic progression in the colon. Cancer 75:2818-26.

109. Senger, D. R., L. F. Brown, C. A. Perruzzi, A. Papadopoulos-Sergiou, and L. Van de Water. 1995. Osteopontin at the tumor/host interface. Functional regulation by thrombin-cleavage and consequences for cell adhesion. Ann N Y Acad Sci 760:83-100.

110. Simpson, M. A., J. Reiland, S. R. Burger, L. T. Furcht, A. P. Spicer, T. R. Oegema, Jr., and J. B. McCarthy. 2001. Hyaluronan synthase elevation in metastatic prostate carcinoma cells correlates with hyaluronan surface retention, a prerequisite for rapid adhesion to bone marrow endothelial cells. J Biol Chem 276:17949-57.

111. Sparrow, C. P., H. Leffler, and S. H. Barondes. 1987. Multiple soluble beta-galactoside-binding lectins from human lung. J Biol Chem 262:7383-90.

112. Sponsel, H. T., P. S. Guzelian, S. E. Brown, R. Breckon, C. Ray, F. R. Simon, and R. J. Anderson. 1996. Mechanisms of recovery from mechanical injury of cultured rat hepatocytes. Am J Physiol 271:C721-7.

113. Swaminathan, G. J., D. D. Leonidas, M. P. Savage, S. J. Ackerman, and K. R. Acharya. 1999. Selective Recognition of Mannose by the Human Eosinophil Charcot-Leyden Crystal Protein (Galectin-10): A Crystallographic Study at 1.8 A Resolution. Biochemistry 38:15406.

114. Tammi, R., K. Rilla, J. P. Pienimaki, D. K. MacCallum, M. Hogg, M. Luukkonen, V. C. Hascall, and-M. Tammi. 2001. Hyaluronan enters keratinocytes by a novel endocytic route for catabolism. J Biol Chem 276:35111-22.

115. Truong, M. J., V. Gruart, J. P. Kusnierz, J. P. Papin, S. Loiseau, A. Capron, and M. Capron. 1993. Human neutrophils express immunoglobulin E (IgE)-binding proteins (Mac-2/epsilon BP) of the S-type lectin family: role in IgE-dependent activation. J Exp Med 177:243-8.

116. Tsuchiyama, Y., J. Wada, H. Zhang, Y. Morita, K. Hiragushi, K. Hida, K. Shikata, M. Yamamura, Y. S. Kanwar, and H. Makino. 2000. Efficacy of galectins in the amelioration of nephrotoxic serum nephritis in Wistar Kyoto rats. Kidney mnt 58:1941-52.

117. Tuxhorn, J. A., G. E. Ayala, and D. R. Rowley. 2001. Reactive stroma in prostate cancer progression. J Urol 166:2472-83.

118. Unger, G., G. Adams, A. Davis, and K. Ahmed. 2002. Effective chemotherapeutic activity by sub50-nm nanoparticle antisense to protein kinase CK2 for eradication of in vivo tumor nests via targeted caveolar-mediated endocytosis, p. 577, vol. 43. ACRR Proceedings.

119. van den Brule, F. A., D. Waltregny, F. T. Liu, and V. Castronovo. 2000. Alteration of the cytoplasmic/nuclear expression pattern of galectin-3 correlates with prostate carcinoma progression. Int J Cancer 89:361-7.

120. Veronese, F. M., B. Sacca, P. Polverino de Laureto, M. Sergi, P. Caliceti, O. Schiavon, and P. Orsolini. 2001. New PEGs for peptide and protein modification, suitable for identification of the PEGylation site. Bioconjug Chem 12:62-70.

121. Wang, L., H. Inohara, K. J. Pienta, and A. Raz. 1995. Galectin-3 is a nuclear matrix protein which binds RNA. Biochem Biophys Res Commun 217:292-303.

122. Wang, T. N., X. H. Qian, M. S. Granick, M. P. Solomon, V. L. Rothman, and G. P. Tuszynski. 1995. The effect of thrombospondin on oral squamous carcinoma cell invasion of collagen. Am J Surg 170:502-5.

123. Wirth, T., E. Soeth, F. Czubayko, and H. Juhl. 2002. Inhibition of endogenous carcinoembryonic antigen (CEA) increases the apoptotic rate of colon cancer cells and inhibits metastatic tumor growth. Clin Exp Metastasis 19:155-60.

124. Xie, R. L., and G. L. Long. 1995. Role of N-linked glycosylation in human osteonectin. Effect of carbohydrate removal by N-glycanase and site-directed mutagenesis on structure and binding of type V collagen. J Biol Chem 270:23212-7.

125. Yang, Q. S., K. Ying, H. L. Yuan, J. Z. Chen, X. F. Meng, Z. Wang, Y. Xie, and Y. M. Mao. 2002. Cloning and expression of a novel human galectin cDNA, predominantly expressed in placenta(1). Biochim Biophys Acta 1574:407-11.

126. Yang, R.-Y., D. Hsu, and F.-T. Liu. 1996. Expression of galectin-3 modulates T-cell growth and apoptosis. Proc Natl Acad Sci USA 93:6737-6742.

127. Yang, R. Y., P. N. Hill, D. K. Hsu, and F. T. Liu. 1998. Role of the carboxyl-terminal lectin domain in self-association of galectin-3. Biochemistry 37:4086-92.

128. Yoshii, T., T. Fukumori, Y. Honjo, H. Inohara, H. R. Kim, and A. Raz. 2002. Galectin-3 phosphorylation is required for its anti-apoptotic function and cell cycle arrest. J Biol Chem 277:6852-7.

129. Yu, F., R. L. Finley, Jr., A. Raz, and H. R. Kim. 2002. Galectin-3 Translocates to the Perinuclear Membranes and Inhibits Cytochrome c Release from the Mitochondria. A ROLE FOR SYNEXIN IN GALECTIN-3 TRANSLOCATION. J Biol Chem 277:15819-27.

130. Zhu, W., H. Sano, R. Nagai, K. Fukuhara, A. Miyazaki, and S. Horiuchi. 2001. The role of galectin-3 in endocytosis of advanced glycation end products and modified low density lipoproteins. Biochem Biophys Res Commun 280:1183-8.

1. A particle for delivering biologically active molecules to a cell and at least one type of biologically active molecule, wherein said particle is a nanoparticle comprising a bioactive component, a surfactant molecule having an HLB value of less than about 6.0 units, a biocompatible polymer, and a cell recognition component, wherein said particle has an average diameter of less than about 200 nanometers as measured by atomic force microscopy following drying of said particle and said biologically active molecule is a galectin.

2. The particle according to claim 1, wherein said bioactive component is selected from the galectin family consisting of galectin-1, galectin-2, galectin-4, galectin-5, galectin-6, galectin-7, galectin-8, galectin-9, galectin-10, galectin-11, galectin-12, galectin-13, and galectin-14, an antibody or a fragment of an antibody that binds to galectin-1, galectin-2, galectin-3, galectin-4, galectin-5, galectin-6, galectin-7, galectin-8, galectin-9, galectin-10, galectin-11, galectin-12, galectin-13, and galectin-14.

3. The particle according to claim 1, wherein said biologically active molecule is N-terminally truncated galectin-3.

4. The particle according to claim 1, wherein said bioactive component is an anti-sense polynucleic acid selected from the group consisting of galectin-1, galectin-2, galectin-3, galectin-3, galectin-4, galectin-5, galectin-6, galectin-7, galectin-8, galectin-9, galectin-10, galectin-11, galectin-12, galectin-13, and galectin-14.

5. The particle according to claim 1, wherein said bioactive component is a polynucleic acid encoding a protein selected from the group consisting of galectin-1, galectin-2, galectin-3, N-terminally truncated galectin-3, galectin-4, galectin-5, galectin-6, galectin-7, galectin-8, galectin-9, galectin-10, galectin-11, galectin-12, galectin-13, and galectin-14.

6. The particle according to claim 1, wherein said cell recognition component is a galectin.

6. The particle according to claim 1, wherein said cell recognition component is a galectin.

7. The particle according to claim 4, wherein said cell recognition component is selected from the group consisting of galectin-1, galectin-2, galectin-3, N-terminally truncated galectin-3, galectin-4, galectin-5, galectin-6, galectin-7, galectin-8, galectin-9, galectin-10, galectin-11, galectin-12, galectin-13, and galectin-14; an antibody or a fragment of an antibody that binds to galectin-1, galectin-2, galectin-3, galectin-4, galectin-5, galectin-6, galectin-7, galectin-8, galectin-9, galectin-10, galectin-11, galectin-12, galectin-13, and galectin-14.

8. A particle for delivering biologically active molecules to a cell and at least one type of biologically active molecule, wherein said particle is a liposome that contains a cell recognition component is a galectin.

9. The particle according to claim 8, wherein said galectin is selected from the group consisting of galectin-1, galectin-2, galectin-3, N-terminally truncated galectin-3, galectin-4, galectin-5, galectin-6, galectin-7, galectin-8, galectin-9, galectin-10, galectin-11, galectin-12, galectin-13, and galectin-14, an antibody or a fragment of an antibody that binds to galectin-1, galectin-2, galectin-3, galectin-4, galectin-5, galectin-6, galectin-7, galectin-8, galectin-9, galectin-1 0, galectin-11, galectin-12, galectin-13, and galectin-14.

10. A particle for delivering biologically active molecule to a cell and at least one type of biologically active molecule, wherein said particle is a liposome and said biologically active molecule is a galectin.

11. The particle according to claim 10, wherein said galectin is selected from the group consisting of galectin-1, galectin-2, galectin-3, galectin-4, galectin-5, galectin-6, galectin-7, galectin-8, galectin-9, galectin-10, galectin-11, galectin-12, galectin-13, and galectin-14; an antibody or a fragment of an antibody that binds to galectin-1, galectin-2, galectin-3, galectin-4, galectin-5, galectin-6, galectin-7, galectin-8, galectin-9, galectin-10, galectin-11, galectin-12, galectin-13, and galectin-14.

12. The particle according to claim 10, wherein said biologically active molecule is N-terminally truncated galectin-3.

13. A method of treating disease by administering to a patient in need a particle as set forth in claim 1.

14. A method of treating disease by administering to a patient in need a particle as set forth in claim 8.

15. A method of treating disease by administering to a patient in need a particle as set forth in claim 10.
